(19) United States
(12) Patent Application Publication
(10) Pub. No.: US 2012/0029216 A1
(43) Pub. Date: Feb. 2, 2012

(54) SCALABLE SYNTHETIC PROCESS FOR MAKING TERAMEPROCOL

(75) Inventors: Qingqi Chen, Chapel Hill, NC (US); Jessica Andrea Blomburg, Chapel Hill, NC (US)

(73) Assignee: Erimos Pharmaceuticals LLC, Houston, TX (US)

(21) Appl. No.: 13/056,733

(22) PCT Filed: Jul. 31, 2009
(86) PCT No.: PCT/US09/52465

§ 371 Date: Apr. 13, 2011

Related U.S. Application Data
(60) Provisional application No. 61/085,511, filed on Aug. 1, 2008.

(51) Int. Cl. C07C 41/30; C07D 307/42

(52) U.S. Cl. 549/502; 568/644

(57) ABSTRACT

A manufacturing process for making terameprocol (1) which includes the following reaction scheme, wherein a first general reaction is the formation of a furan intermediate (39) and a second general reaction is the ring-reduction and ring-opening of the furan intermediate (39) to form the terameprocol (1):

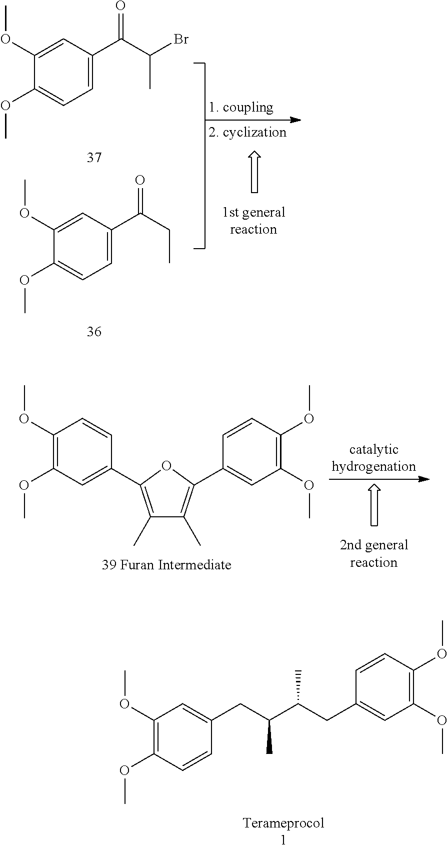

### CROSS-REFERENCE TO RELATED APPLICATIONS

This application claims priority to PCT application number PCT/US2009/052465 filed on Jul. 31, 2009 which claims priority to Unites States provisional patent application No. 61/085,511, filed on Aug. 1, 2008, the contents of which are expressly incorporated herein.

### BACKGROUND OF THE INVENTION

Terameprocol 1, also know as M₄N, is tetra-O-methyl nordihydroguaiaretic acid, a semi-synthetic derivative of nordihydroguaiaretic acid (NDGA, 2).

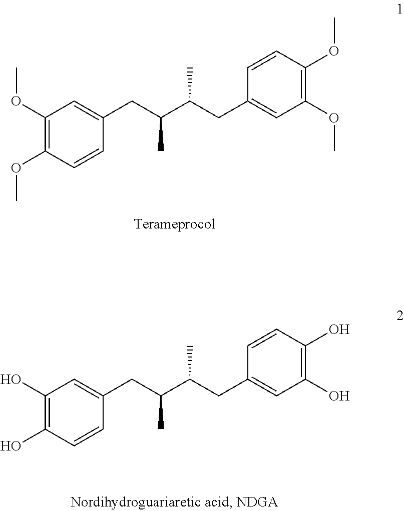

Terameprocol is designed to target abnormal tumor cells while causing little or no toxicity to healthy cells. Working at the DNA level, terameprocol has a mechanism of action that inhibits or prevents the production and activation of survivin, a protein that is produced excessively in tumor cells, thus preventing cell replication and enhancing the body's ability to eliminate abnormal cells through cell death, or apoptosis.

### BRIEF SUMMARY OF THE INVENTION

The present invention relates to a manufacturing process for making terameprocol 1 which comprises the following reaction scheme, wherein a first general reaction is the formation of a furan intermediate 39 and a second general reaction is the ring-reduction and ring-opening of the furan intermediate 39 to form the terameprocol 1 (Scheme 13):

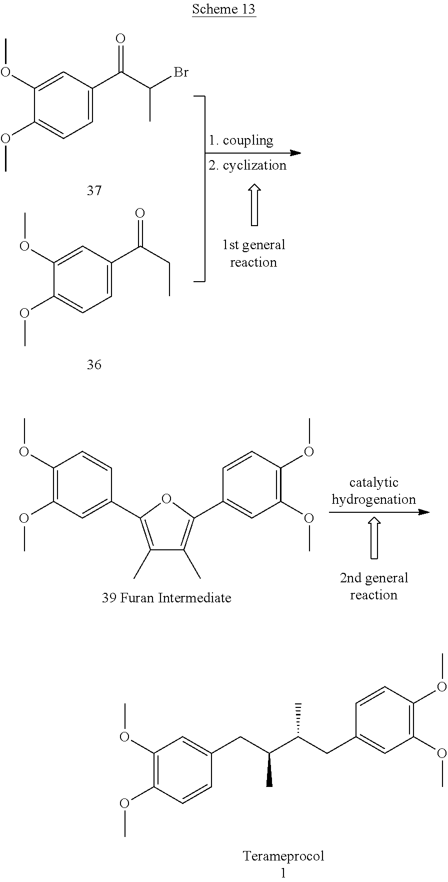

The first general reaction to form the furan intermediate 39 is a two-reaction, one-purification process, in which the first reaction is a coupling reaction, in which a ketone-catechol compound 36 is treated by an organic basic catalyst, followed by reaction with a bromide-ketone-catechol compound 37 to give a corresponding diketone intermediate, and in which the second reaction is a cyclization reaction, in which the diketone intermediate is converted to the furan intermediate 39.

The organic basic catalyst for the coupling reaction of the ketone-catechol compound (36) with the bromide-ketone-catechol compound (37) preferably is an alkali metal salt of an alkyl alcohol having a formula MOR, in which M is an alkali metal ion selected from the group consisting of K⁺, Na⁺ and Li⁺, and R is a linear or branched saturated hydrocarbon chain having 4 to 10 carbon atoms; the amount of the basic catalyst used preferably is about 0.5 to about 1.5 molar equivalents of compound (36); the molar ratio of compound (37) to compound (36) preferably is about 0.5 to about 1.7; and a solvent system preferably is used in the coupling reaction, wherein the solvent system preferably is a single solvent or a mixture of two solvents selected from the group consisting of tetrahydrofuran, 1,2-dimethoxyethane, 1,3-dimethoxypropane, and dimethyl formamide.

The reaction temperature for the coupling reaction preferably is about −30° C. to about −70° C., and the temperature for the cyclization reaction is about 55° C. to about 65° C.

The catalyst for the second general reaction preferably is a mixture of two types of palladium catalysts, one being favorable for furan ring-reduction and the other being favorable for a ring-opening reaction, in which the palladium catalysts preferably contain about 40 to about 60% water, and on a dry basis, about 5% to about 20% palladium, and about 80% to about 95% active carbon silica alumina gel.

A preferred catalyst favorable for furan 39 reduction is selected from the group consisting of a catalyst having 10% Pd on carbon, 5% Pd on SiO₂-Al₂O₃. The catalyst loading amount is favorably 1 mol %˜4 mol % of furan 39.

### DETAILED DESCRIPTION OF THE PREFERRED EMBODIMENTS

More specifically, the process of the present invention relates to a synthetic process for manufacturing terameprocol 1 as shown in the following Scheme 14:

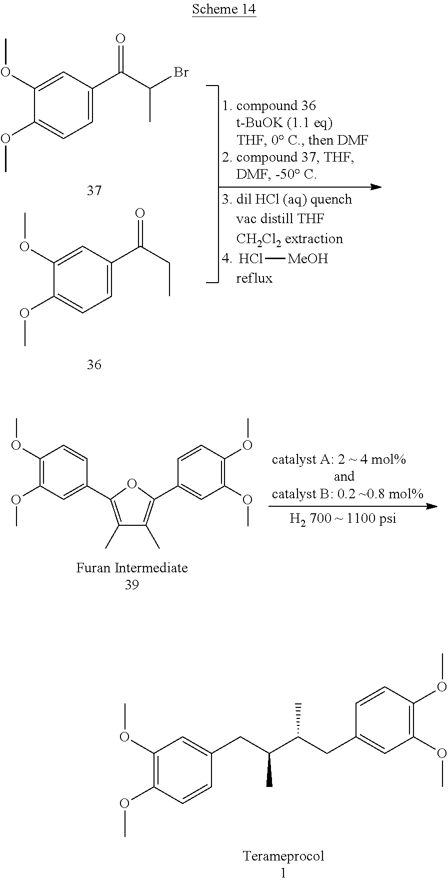

The process starts with compound 36 and compound 37. The first general step is the preparation of the furan intermediate 39, and step 2 is the preparation of terameprocol. The key intermediate compound 39 is prepared with about 90% to about 95% yield by coupling the ketone-catechol 36 with the bromide-ketone-catechol 37. The first general step, the synthesis of compound 39, is a two-step reaction and a one-purification process, in which the coupling of 36 with 37 first gives the diketone 38 (Scheme 10) using potassium tert-butoxide (t-BuOK) as a basic catalyst. Without purification, compound 38 is cyclized to give the furan 39 under acidic conditions. After ring-reduction and ring opening under H₂ pressure of 700 psi ˜1100 psi, the furan intermediate 39 is converted to terameprocol 1 in 55% yield. The second general step uses a combination of two catalysts, i.e., catalyst A and catalyst B, in which catalyst A is favorable for the ring-reduction and catalyst B is favorable for ring-opening, and both of which work together to facilitate the formation of terameprocol.

Among many literature-based procedures for the synthesis of terameprocol, the synthetic route shown in Scheme 10 above, as reported by Perry et al. (1972) and U.S. Pat. No. 3,906,004, both supra, is attractive, based on the following characteristics: (1) inexpensive starting materials; (2) good yields for most synthetic steps; (3) less steps in the synthetic route; (4) the catalytic hydrogenation only produces the desired meso-form conformation of terameprocol, which allows the purification process to be easy and convenient; and (5) all synthetic steps have strong literature precedents.

However, the synthetic procedure of Perry et al's (as outlined in Scheme 10) remains to be optimized for scalability and reproducibility for several reasons.

First, the preparation of the diketone intermediate 38, used liquid ammonia as a solvent, and more than one molar equivalent of sodium was used to generate in situ sodium amide as a basic catalyst. A larger volume of liquid ammonia and a larger amount of sodium are difficult to handle in a larger scale production process. The reproducibility for this reaction is also an issue. When the inventor attempted to prepare the diketone compound 38 using similar conditions (such as (a) Na/NH₃/FeCl₃, −30° C.; (b) NaNH₂, −30° C.; (c) lithium diisopropylamide (LDA)/THF, −40° C.; (d) LiNH₂, −30° C.; (e) LDA/THF, −40° C.), the considerable amount of the epoxide compound 47 observed, which was also reported by Perry et al (1972). Compound 47 was presumably formed through a six-member ring transit-state intermediate 46, where metal ion Li⁺ (or Na⁺) is chelated with two oxygen atoms of the ketone group. The neighbor bromide is then detached, which is favorable for the formation of the epoxide (Scheme 15).

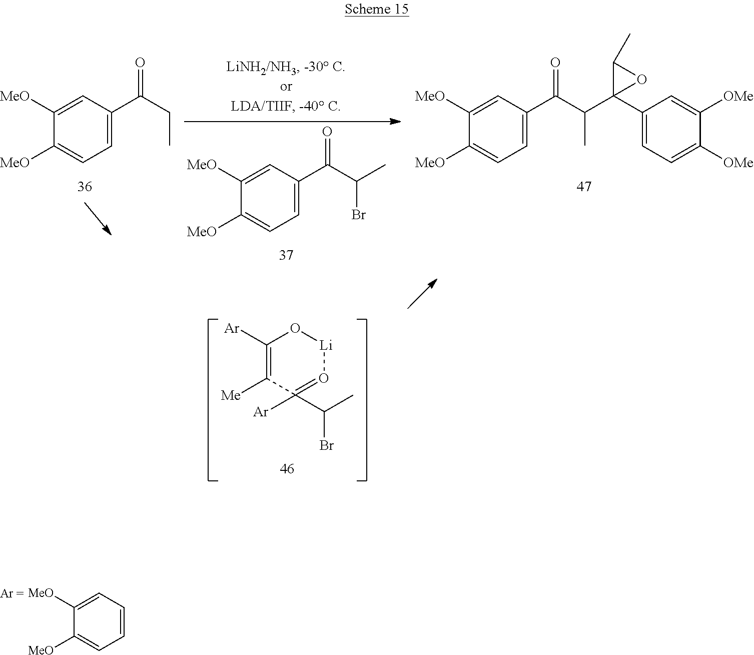

Second, the preparation of terameprocol needed to use an expensive palladium oxide (PdO) catalyst with high loading (20% to 81% mol equivalent), and high pressure (1500 psi). Palladium oxide is an expensive catalyst, and higher catalyst loading will contribute to a much higher manufacturing cost. According to the results reported Perry et al. (1972), supra, only fresh and finely powdered palladium oxide worked well for this reaction (Scheme 16), which is not convenient for an industrial manufacturing process.

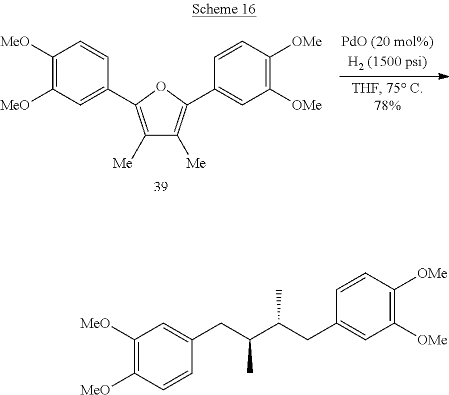

Although palladium chloride (20% mol equivalent was needed) was used for the conversion of compound 39 to terameprocol 1 with 79% yield as reported (Perry et al. (1972), supra), the present inventor consistently obtained only 20% to about 35% of the expected product with about 50% side products including the partially reduced THF-intermediate 40 (Scheme 10).

Having identified key parameters in the synthetic process of terameprocol, the present inventor focused efforts on the following: (1) as scalable and convenient synthetic method for the furan intermediate 39; and (2) a scalable method for the conversion of the furan intermediate 39 to terameprocol 1 with lower catalytic loading so as to reduce the manufacturing cost. The process of the present invention comprises two general reactions: first, synthesis of the furan intermediate 39, and second, ring opening and reduction to make terameprocol (Scheme 13).

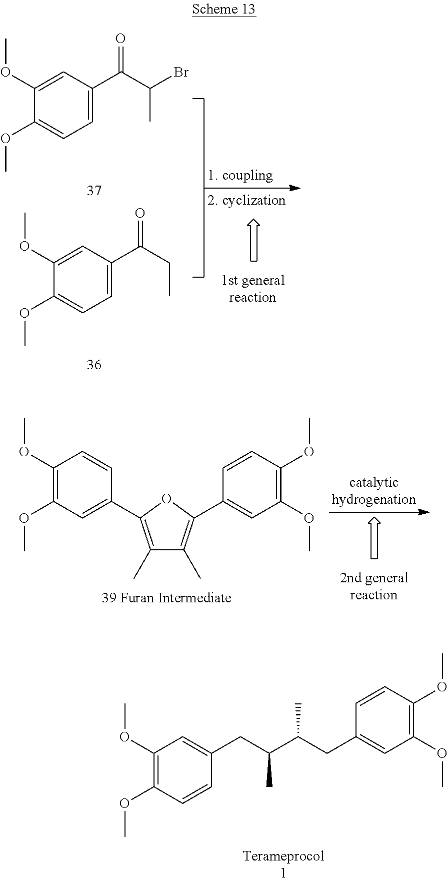

### 1. Process Optimization for 1^{st} General Reaction: Coupling and Cyclization

The primary goal of process optimization of the alkylation and cyclization steps was to improve yields and streamline the developed reactions. In the present invention, the preparation of the diketone intermediate and the subsequent cyclization step to the furan intermediate 39 could be combined without isolating the diketone intermediate 38. Combining these steps shortens the processing time and increases the overall yield.

For this approach to be successful, a common solvent for both steps was explored and toluene was identified as a potentially viable solvent for both steps. The alkylation of propiophenone 36 was performed using t-BuOK in toluene-dimethylformamide (DMF) to give diketone intermediate (Scheme 17).

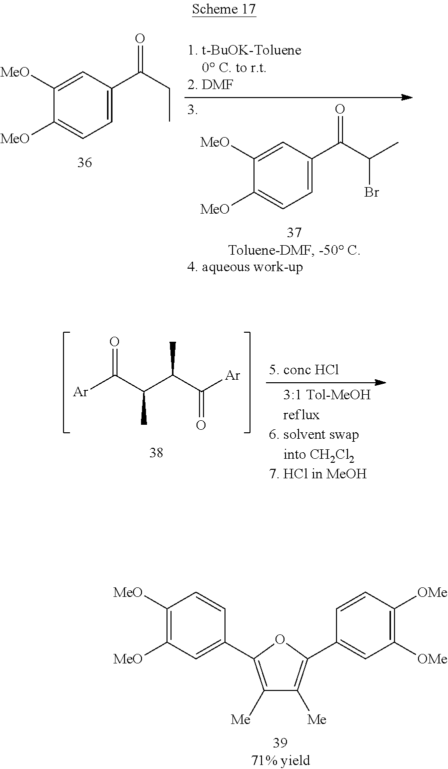

When the coupling was complete, the reaction underwent a general aqueous work-up procedure, i.e. the reaction mixture was simply placed in the separated funnel, and was washed with water to remove water soluble materials. Unfortunately, once DMF was removed by water washes, diketone 38 was not completely soluble in toluene, resulting in a messy work-up (i.e., the formation of an emulsion and poor separation of the aqueous phase). The diketone-rich toluene was diluted with MeOH, treated with concentrated HCl, and heated to reflux for 1 hour. The reaction appeared to stop at 50% conversion. In order to drive the reaction to completion, the reaction mixture was concentrated, diluted with CH₂Cl₂, and treated with HCl in MeOH. After 30 min. the reaction was complete (71% isolated yield, 2-steps). Overall, using toluene as a solvent should be avoided due to a poor solubility profile and sluggish reactivity in the cyclization step.

A second paragraph approach toward combining the coupling and cyclization steps involved acidifying and heating the reaction mixture after the coupling step. In this case, the diketone intermediate was prepared using t-BuOK in THF-DMF and quenched with an excess of concentrated HCl followed by heating to reflux (Scheme 18).

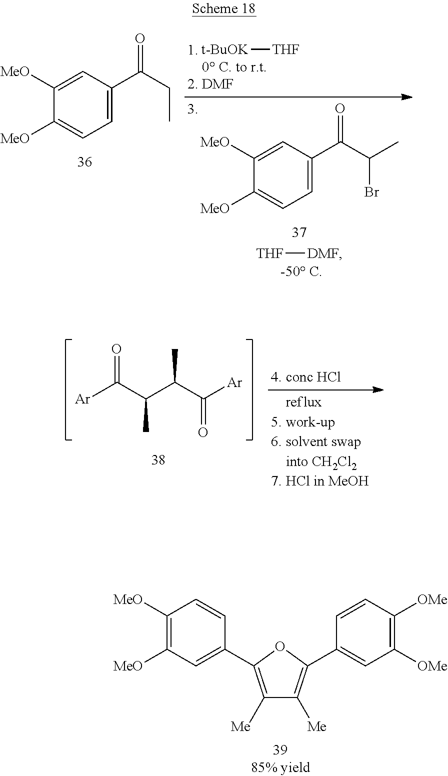

Unfortunately, after refluxing for 2 hours the cyclization appeared to stop at about 50% conversion. Adding additional acid did not appear to push the reaction to completion. It is possible that the presence of large quantities of DMF interferes with the cyclization. The mixture which contained 38 and 39 was recovered after removal of THF through distillation. The residue was then taken up by CH₂Cl₂ and washed with water, which removed DMF. The organic phase was separated and concentrated. This concentrated solution contained 38 and 39 was then treated with HCl in MeOH at reflux, the reaction was complete within 15 min. (85% isolated yield).

To improve the process, DMF was removed prior to the cyclization step. The diketone intermediate 38 is highly soluble in CH₂Cl₂, which appeared to be an ideal solvent to bring the diketone into the cyclization step. In one experiment, the starting propiophenone 36 (10 g) in THF was added to 25 wt % t-BuOK in THF at 10° C. (Scheme 19). The mixture was warmed to room temperature, DMF was added to dissolve the resulting suspension, and the mixture was cooled to −50° C. A solution of α-bromoketone 7 in 3:1 THF-DMF was added dropwise and the reaction was complete within 1 hour.

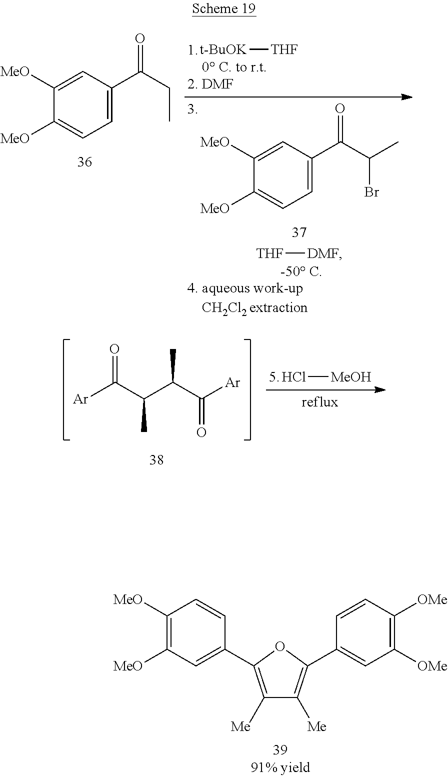

The work-up involved a quench with 1N HCl, removing the bulk of THF by distillation, and extracting diketone 38 into CH₂Cl₂. After washing with water to remove residual DMF, the diketone-rich organic solution was concentrated to remove about 75% of solvent without implementing a discrete drying step. The solution was heated to reflux and treated with 3% HCl in MeOH at reflux. After 15 min, the product slurry was gradually cooled to 5° C. and filtered. As originally anticipated, the streamlined approach was shown to be time efficient and produced a high yield. Furan intermediate 39 was obtained with 91% w/w isolated yield, which was a significant improvement compared to the 78% and 88% isolated yields obtained, respectively, for the individual coupling and cyclization steps.

During scale-up on 170 g propiophenone 36, the process performed as expected using equimolar amounts of propiophenone 36, α-bromoketone 37 and t-BuOK (Scheme 20).

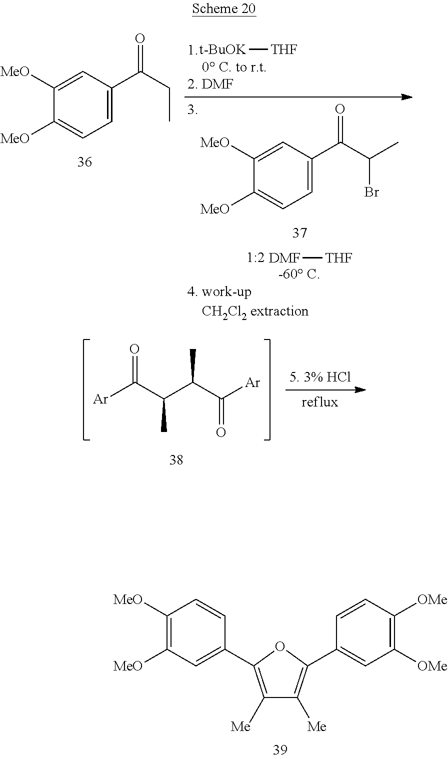

Based on the batch size, cooling the mixture to −70° C. was necessary so as to maintain an internal reaction temperature of about −55° C. to about −60° C. The diketone, under this condition, was formed exclusively (i.e., epoxide impurity 47 was not observed by liquid chromatography (LC) or mass spectroscopy (MS)). After CH₂Cl₂ extracting and the aqueous work-up as described for Scheme 19, the solution of diketone 38 was concentrated and treated with 3% HCl in MeOH. Crystallization of furan 39 was hampered by the presence of excess CH₂Cl₂. To address this problem, excess CH₂Cl₂ was distilled after the HCl-MeOH addition. Once the CH₂Cl₂ was removed, crystallization occurred rapidly and the reaction was complete within 1 hour. Furan 39 was isolated as a white solid in excellent yield (92%) and high purity (>97%).

TABLE A
Scale Up Result of Synthesis Of Furan 39

| Propiophenone 36 (g) | Furan 39 (g) | Yield (%) | Purity (%) |
|---|---|---|---|
| 170 | 286 | 92 | >97 |

### Conclusion

The alkylation/cyclization sequence was successfully demonstrated on large scale (170 g). In addition to being high-yielding, this approach also proved to be time-efficient. Although the entire sequence could be carried out within 8 hours, it was notable that the diketone solution in CH₂Cl₂ could be held at room temperature for up to 1 week without signs of decomposition (as determined by LC and MS), indicating a possible hold point.

Based on experimental observations, it is highly recommended to maintain efficient cooling during the α-bromoketone addition. On a larger scale, the reaction mixture was cooled to −70° C. in order to maintain a batch temperature of −60° C. to −55° C., while allowing a short addition time (22 min.). It is also recommended to remove as much CH₂Cl₂ as possible prior to adding HCl-MeOH, in order to allow furan 39 to precipitate from solution.

### 2. Process Optimization for the 2^{nd} General Reaction: Hydrogenation

Development of furan 39 hydrogenation continued on larger scale (3 g). For these reactions, a 500 mL vessel was used, which accommodated a larger magnetic stirbar for better stirring. In a typical experiment, about 3 g furan 39, Pd/C (2.5 mol % Pd), and various amounts of 2-ethylhexanoic acid were used in 15 mL/g of solvent. The reaction mixture components were combined in a hydrogenator. The vessel was sparged with N₂ and pressurized with H₂ (pressure: 300 psi ˜400 psi) and vented (3 times), which ensured the vessel was filled with pure H₂. The vessel was then pressurized with H₂ (pressure: 300 psi 400 psi), then placed in a preheated oil bath. To sample the reaction, the vessel was cooled to 18° C., vented and the vessel was opened and the sample was withdrawn via pipette. The hydrogenation was then continued as described above. The general strategy for the development work was to perform the hydrogenation at temperatures preferred between 70° C.˜110° C. and at pressures preferred between 700 psi ˜1310 Psi H₂. The overall goal was to find conditions that would convert all tetrahydrofuran intermediate (THF Int) 40 to products and to minimize or eliminate the formation of impurity 48 (formed via cyclization of terameprocol) as shown in Scheme 21.

The results are shown in Table B, development of the furan 39 hydrogenation step using 10% Pd/C (50% wet, Degussa type E101 NE/W [Note: this catalyst is available from Sigma-Aldrich, which contains 50% water w/w, and the dry form is as 10% Palladium and 90% activated carbon powder. The carbon powder particle size is 20 micron]) in 15 mL/g solvent in the presence of 2-ethyl hexanoic acid (2-EHA) on a 3 g scale.

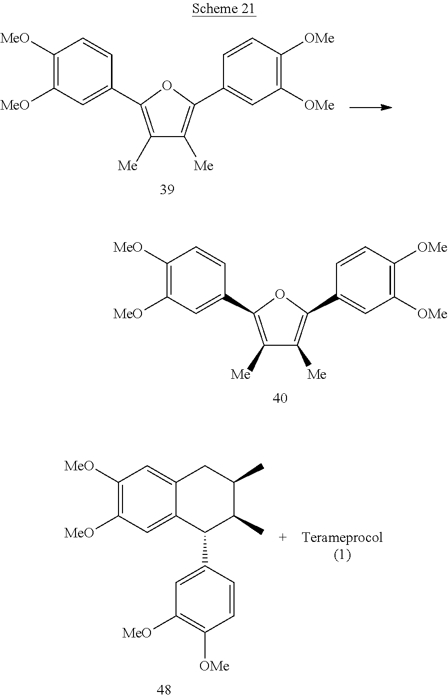

TABLE B

| | | 2-EHA | Temp | P (psi) | Time | Furan | THF Int | Impurity | Terame |
|---|---|---|---|---|---|---|---|---|---|
| Entry | Solvent | (eq) | (° C.) | | (h) | 39 (%) | 40 (%) | 48 (%) | p. 1 (%) |
| 1 | EtOAc | 0.1 | 94 | 1100 | 20 | 0 | 34 | 25 | 41 |
| 2 | Heptane | 0.1 | 100 | 1125 | 20 | 90 | 10 | 0 | 0 |
| 3 | IPAc* | 0.05 | 102 | 1250 | 21 | 0 | 36 | 20 | 44 |
| 4 | 7% v/v & IPAc | | 101 | 1250 | 16 | 0 | 16 | 30 | 54 |
| | IPA** | | | | | | | | |
| 5 | IPAc | 2.3 | 108 | 1250 | 5 | 0 | 57 | 12 | 31 |
| 6 | 10% IPA-IPAc | 0.05 | 106 | 250 | 15 | 0 | 41 | 21 | 38 |
| | | | | (leak) | | | | | |
| 7 | 5% IPA-IPAc | 0.1 | 100 | 1200 | 23 | 0 | 57 | 13 | 30 |
| 8 | 30% IPA-IPAc | 0.25 | 105 | 1200 | 18 | 0 | 0 | 35 | 65 |

*IPAc = isopropyl acetate
**IPA = isopropanol

Although ethyl acetate showed some promise on a small scale, the scaled-up reaction stalled after 20 hours (Table B, entry 1). Therefore, EtOAc was no longer considered for scale-up. Despite promising results on a smaller scale, furan 39 was not significantly hydrogenated on a larger scale when heptane was used at 1125 psi, suggesting that higher pressures may be needed (entry 2). Next, isopropyl acetate (IPAc) was considered due to its lower polarity and higher boiling point (entry 3). The reaction gave similar results to EtOAc, giving a stalled reaction after 21 hours. Adding 7% (v/v) isopropanol (IPA) to the stalled reaction mixture greatly improved conversion of THF intermediate 40 to product, but increased impurity 48 formation was also observed (entry 4). Isopropyl acetate was tried again, this time using more 2-ethylhexanoic acid instead of isopropanol (entry 5). Encouragingly, less impurity 48 formed in the absence of isopropanol; however the reaction stalled after 5 hours. Therefore, 10% isopropanol was added up front to the isopropyl acetate mixture with the expectation that this would speed the reaction (entry 6). Unfortunately, the reactor leaked after pressurizing to about 1200 psi, which was reduced to 250 psi over a 15 hour period. Encouragingly, however, despite the slow leak, significant conversion of starting furan 39 to the all-cis-tetrahydrofuran 40, impurity 48 and terameprocol 1 was also observed. Even with incomplete conversion, it was noted that a significant amount of impurity 48 formed. Reducing the amount of isopropanol to 5% (v/v) was found to reduce the amount of impurity 48, however the reaction stalled (entry 7). In another attempt, the 30% isopropanol in isopropyl acetate was employed (entry 8). After 18 hours, the reaction reached completion. Although a 1.8:1 ratio of terameprocol 1 to impurity 48 was observed by LC and MS analysis, the impurity 48 could be effectively removed using heptane as the isolation and crystallization solvent. In the final experiment, terameprocol was isolated in 44% w/w yield as determined by liquid chromatography (LC).

With a viable hydrogenation procedure in place, a scale-up was performed on 230 g of furan 39 in an 8 L Parr hydrogenation vessel equipped with an overhead magnetic stir drive. The hydrogenation was performed using 15 mL/g 30% isopropanol in isopropyl acetate, 2.5 mol % 10% Pd/C (Degussa type E101 NE/W), and 25 mol % 2-ethylhexanoic acid. The batch temperature was maintained between 100° C. and 110° C. and the pressure was maintained between 1230 psi H₂ and 1310 psi H₂. After 16 hours, the pressure decreased from 1310 psi to about 1200 psi and the reaction appeared to stall; however, charging additional catalyst 10% Pd/C (Degussa type E101 NE/W) and re-pressurizing to 1310 psi allowed the reaction to reach completion.

The reaction was worked up by carefully filtering off Pd/C through Celite® filter material and solvent exchanging into heptane. Terameprocol 1 was isolated in lower than anticipated yield (23.8% w/w yield), after crystallizing from heptane and drying at 50° C. in vacuo. By LC/MS, the purity was found to be >99%.

Conclusions:

The key issue with the hydrogenation step is the low yield, which is due in large part to the formation of cyclized impurity 48. Improving yield hinges around reducing the formation of the impurity. It is proposed that this can be achieved by focusing efforts on less polar solvents. Based on experimental results, the use of moderately strong acids (i.e., AcOH) generated a significantly higher amount of impurity 48, compared with a weaker acid (i.e., 2-ethylhexanoic acid). The amount of acid used did not appear to have a major impact on formation of impurity 48. Although the hydration may be accelerated by the presence of a proton source, the reaction is still viable in the absence of an acid. Alternative organic or inorganic acids weaker than 2-ethylhexanoic acid is also possible, which was determined by additional studies. In addition, a single solvent system using a Pd catalyst on alternate supports (different types of carbon, charcoal, alumina, silica, etc.) is also possible, which was determined by additional studies.

One of the key difficulties encountered in the hydrogenation development was the use of small-scale equipment that required cooling and opening to the air in order to sample for analysis (i.e., the equipment did not have a sampling port). This presented a problem in some cases when significant amounts of starting furan 39 and THF intermediate 40 were present at time of sampling. As the reaction mixture was cooled, these materials precipitated and appeared to coat the carbon catalyst support. Once absorbed onto the carbon catalyst support, it was unknown whether the solid materials actually dissolved on re-heating, or if they continued to coat the catalyst. This may be a key factor in the numerous stalled reactions that were observed.

Therefore, it is preferred that reaction sampling be done when the reaction is hot, especially when the furan 39 is not soluble in the solvent system at room temperature. Alternatively, a solvent in which furan 39 is highly soluble is preferred for further optimization (i.e., THF, CH₂CL₂, CHCl₃, etc.).

Specifically, the yield could be improved if the cyclic impurity 48 can be reduced. Throughput can also be improved by reducing the solvent requirements for the reaction (currently, the 15 L solvent/kg furan 39). As disclosed below, studies were conducted regarding catalyst type and loading and studies were done at higher temperature ranges (>125° C.) in an effort to create some improvement in reaction time and impurity profile.

### 3. Catalyst Screening

To find a better catalyst or a combination of several catalysts, a number of screening experiments were designed for Scheme 20 as set forth above, using the following reaction conditions: 165 mg substrate, 12 mg dry weight catalyst, 30% IPA in isopropyl acetate, 0.018 mL ethyl hexanoic acid, 18 hours reaction time. Experiments were carried out in a HEL ChemSCAN high pressure reactor comprising 8×10 mL stainless-steel reactors with oil-bath heating and rare-earth magnetic followers.

To find a suitable column to analyze and monitor the reaction progress, the present inventor attempted a number of methods, including HPLC and GC-MS. It was found that GC-MS with a Zorbax™ MS-5 column (Agilent Technologies Inc.) achieved good separation of all identified components. The results of the experiments are shown in Table C:

TABLE C
Catalyst Screening (temperature: 100° C., pressure: 90 bar, time: 3 hours)

| | | Furan 39 | THF INT | Impurity | Teramep 1 | Ring hydrog |
|---|---|---|---|---|---|---|
| | Catalyst* | (%) | 40 (%) | 48 (%) | (%) | Byprod (%). |
| 1 | A501023-10 | 17.3 | 81.5 | 0 | 0 | 1.2 |
| 2 | B103018-5 | 77.6 | 19.6 | 0 | 2.8 | 0 |
| 3 | E101 NE/W GG | 0 | 82.2 | 4.5 | 9.9 | 3.4 |
| 4 | A470129-10 | 0 | 8.9 | 41.9 | 41.3 | 7.8 |
| 5 | A402028-10 | 0 | 2.5 | 42.7 | 49.8 | 5.0 |
| 6 | 10R39 | 0.9 | 32.0 | 24.4 | 37.8 | 4.9 |
| 7 | E101 NE/W GG | 2.4 | 69.9 | 5.7 | 14.4 | 7.7 |
| 8 | 10R39 | 0 | 0.0 | 44.8 | 48.5 | 6.8 |

*Note:
E101 NE/W GG is a 10% Pd/C catalyst obtained from Sigma-Aldrich Inc. The rest catalysts are also 10% Pd/C catalysts, and were obtained from Johnson Matthey Inc. Although they are all 10% Pd/C catalysts, different catalysts have different supporting materials regarding to the particle size, surface area, water percentage, etc.

The results in Table C show the attempt to reproduce results with 10% Pd/C (E101 type, which contains 50% water w/w, the dry form is made as 10% palladium plus 90% activated carbon at 20 micron). These appears to be a significant difference in the activities of these catalysts. The E101 catalyst gives a lot of intermediate 40 in both reactions (entries 3 and 7). Most of the Johnson Matthey catalysts give better conversion than E101 NE/W catalyst but the selectivity of product in relation to byproduct was not good (entries 1, 2, 4, 6, 8).

Experiments were also performed to determine the effect of using different solvents in Scheme 21. The following reaction conditions were used: 165 mg substrate, 2.5 mol % Pd (10R39, 10% Pd/C obtained from Johnson Matthey Inc.), 5 mL solvent, 3 hours reaction time. The results are shown in Table D-1:

TABLE D-1
Solvent Screening (Catalyst: 10R39, temperature:
100° C., pressure: 90 bar, time: 3 hours)

| | | | | | | Ring |
|---|---|---|---|---|---|---|
| | | | | | | hydrog |
| | | Furan 39 | THF Int | Impurity | Teramep | Byprod |
| Entry | Solvent | (%) | 40 (%) | 48 (%) | 1 (%) | (%) |
| 9 | THF | 49.1 | 50.9 | 0.0 | 0.0 | 0.0 |
| 10 | 2-Methyl-THF | 27.9 | 72.1 | 0.0 | 0.0 | 0.0 |
| 11 | Toluene | 7.8 | 74.3 | 3.3 | 14.7 | 0.0 |
| 12 | Propan-2-ol | 0.0 | 38.2 | 36.9 | 24.9 | 0.0 |
| 14 | 2-dimethoxy- | 0.0 | 10.2 | 54.8 | 32.2 | 0.0 |
| | ethanol | | | | | |
| 15 | DMF | 95.7 | 3.5 | 0.5 | 0.3 | 0.0 |
| 16 | Ethyl acetate | 2.7 | 91.0 | 3.0 | 3.3 | 0.0 |

Further experiments were conducted regarding different solvents using a different catalyst in Scheme 21. The following conditions were used: 165 mg substrate, 2.5 mol % Pd (10R39, 10% Pd/C obtained from Johnson Matthey Inc.), 5 mL solvent, no acid, 18 hours reaction time. The results are shown in Table D-2:

TABLE D-2
Solvent Screening (Catalyst: 10R39, temperature:
100° C., pressure: 90 bar, time: 18 hours)

| | | Furan 39 | THF Int | Impurity | Teramep | Ring hydrog |
|---|---|---|---|---|---|---|
| | Solvent | (%) | 40 (%) | 48 (%) | 1 (%) | Byprod (%) |
| 17 | THF | 8.3 | 91.7 | 0 | 0 | 0 |
| 18 | 2-Methyl THF | 2.6 | 91.7 | 1.8 | 1.8 | 2.2 |
| 19 | Toluene | 0.3 | 85.7 | 2.9 | 9.5 | 1.5 |
| 20 | IPA | 0 | 0 | 46 | 36.4 | 17.6 |
| 21 | H₂O | | | | | |
| 22 | 2-Dimethoxy- | 0 | 2.4 | 64.1 | 33.5 | 3.1 |
| | ethanol | | | | | |
| 23 | DMF | 85.9 | 8.7 | 0.7 | 0.4 | 4.4 |
| 24 | Ethyl acetate | 2.8 | 88.8 | 2.5 | 3.8 | 2.1 |

Tables D-1 and D-2 show a comparative study of selected solvents using the best catalyst from Table C (10% Pd/C, type 10R39, available from Johnson Matthey Inc.) after 3 hours and 18 hours respectively. IPA and dimethoxyethanol gave good conversion but selectivity under these conditions was slightly inferior to those using the standard solvent (30% IPA/IP acetate).

Further experiments were conducted regarding different catalysts in Scheme 21. The following reaction conditions were used: 330 mg substrate, 24 mg dry weight catalyst, 30% IPA in isopropyl acetate, 0.036 mL ethyl hexanoic acid, 18 hours reaction time. The results are shown in Table E:

TABLE E
Catalyst Screening (temperature:
100° C., pressure: 90 bar, time: 18 hours)

| | | Furan 39 | THF Int | Impurity | Teramep | Ring hydrog |
|---|---|---|---|---|---|---|
| | Catalyst* | (%) | 40 (%) | 48 (%) | 1 (%) | Byprod (%) |
| 25 | 10R39 | 0 | 1.0 | 27.8 | 41.4 | 29.8 |
| 26 | 10R394 | 0 | 15.5 | 26.4 | 53.2 | 5.0 |
| 27 | E101 NE/W GG | 2.4 | 0.9 | 38.2 | 44.8 | 13.7 |
| 28 | 5% Pd/ | 2.9 | 94.5 | 0.0 | 0.0 | 2.7 |
| | SiO₂—Al₂O₃ | | | | | |
| 30 | E101023-4/1 | 1.1 | 86.4 | 1.4 | 7.2 | 3.9 |
| 31 | E101 NE/W GG | 1.5 | 57.1 | 10.4 | 26.5 | 4.6 |
| 32 | 10R39 | 0 | 1.2 | 41.2 | 49.8 | 7.7 |

*Note:
E101 NE/W GG is a 10% Pd/C catalyst obtained from Sigma-Aldrich Inc. The rest catalysts are also 10% Pd/C catalysts, and were obtained from Johnson Matthey Inc. Although they are all 10% Pd/C catalysts, different catalysts have different supporting materials regarding to the particle size, surface area, water percentage, etc.

In Table E some better selectivity results were seen with product: byproduct ratios of about 2 for catalyst 10R394 (entry 26) at 84% conversion. The silica-alumina supported Pd sample and the mixed-metal E101023 catalyst (entry 28) show conversion only to the THF compound 40. Testing of an alternative sample of E101 NE/W GG (entry 27) showed full conversion but selectivity was only about 50%.

Further experiments were conducted regarding different catalysts in Scheme 21. The following reaction conditions were used: 330 mg substrate, 24 mg dry weight catalyst, 30% IPA in isopropyl acetate, 0.036 mL ethyl hexanoic acid, 18 hours reaction time. The results are shown in Table F:

TABLE F
Catalyst Screening (temperature:
100° C., pressure: 90 bar, time: 18 hours)

| | | Furan 39 | THF Int | Impurity | Teramep | Ring hydrog |
|---|---|---|---|---|---|---|
| | Catalyst* | (%) | 40 (%) | 48 (%) | 1 (%) | Byprod (%) |
| 33 | 10R90 | 0 | 59.7 | 8.0 | 28.0 | 4.4 |
| 34 | 10R37 | 0 | 14.3 | 26.1 | 54.6 | 5.0 |
| 35 | 10R37# | 0 | 87.2 | 0.0 | 0.6 | 12.2 |
| 36 | E101 NE/W GG | 0 | 69.8 | 6.1 | 18.7 | 5.3 |
| 38 | 10R39 | 0 | 1.2 | 35.9 | 61.2 | 1.6 |
| 39 | 10R39# | 0 | 0 | 37.5 | 25.6 | 36.9 |
| 40 | A402032-10 | 0 | 33.5 | 10.2 | 49.7 | 6.6 |

*Note:
E101 NE/W GG is a 10% Pd/C catalyst obtained from Sigma-Aldrich Inc. The rest catalysts are also 10% Pd/C catalysts, and were obtained from Johnson Matthey Inc. Although they are all 10% Pd/C catalysts, different catalysts have different supporting materials regarding to the particle size, surface area, water percentage, etc.
#2 mg of 70% H₃PO₄/SiO₂ added

In Table F an array of unreduced Pd catalysts was evaluated giving a wide range of results. The 10R39 catalyst gave better selectivity in this experiment with 1.7:1 ratio of product to byproduct. Addition of an acid catalyst with this catalyst gave much lower selectivity. The A402032-10 catalyst gave god selectivity to the product (4.9:1) but conversion was incomplete at 65%.

Further experiments were conducted regarding different catalysts in Scheme 21. The following reaction conditions were used: 330 mg substrate, 24 mg dry weight catalyst, 30% IPA in isopropyl acetate, 0.036 mL ethyl hexanoic acid, 18 hours reaction time. The results are shown in Table G:

TABLE G
Catalyst Screening (temperature:
100° C., pressure: 90 bar, time: 18 hours)

| | | Furan 39 | THF Int | Impurity | Teramep | Ring hydrog |
|---|---|---|---|---|---|---|
| | Catalyst* | (%) | 40 (%) | 48 (%) | 1 (%) | Byprod (%) |
| 41 | 10R374 | 0.0 | 93.1 | 0.0 | 0.0 | 6.9 |
| 42 | 20R91 | 2.2 | 40.8 | 8.7 | 33.0 | 15.2 |
| 43 | 20R91 | 1.4 | 19.1 | 13.1 | 38.8 | 27.6 |
| 44 | 10R39 | 0.0 | 3.6 | 31.0 | 57.8 | 7.7 |
| 46 | 5% Pd/ | 6.4 | 88.5 | 0.3 | 1.1 | 3.7 |
| | SiO₂—Al₂O₃ | | | | | |
| 47 | A470129-10 | 0.0 | 0.0 | 51.9 | 36.1 | 12.9 |
| 48 | A302011-5 | 2.7 | 79.7 | 0.0 | 0.0 | 17.6 |

*Note:
The catalysts are 10% Pd/C catalysts except where indicated (such as entry 46), and were obtained from Johnson Matthey Inc. Although they are all 10% Pd/C catalysts, different catalysts have different supporting materials regarding to the particle size, surface area, water percentage, etc.

In Table G some alternative 10% Pd/C catalysts and 20% Pd/C catalysts were evaluated with one of these showing a good selectivity (3:1) but incomplete conversion (75%).

Further experiments were conducted regarding different catalysts in Scheme 21. The following reaction conditions were used: 330 mg substrate, 24 mg dry weight catalyst, 30% IPA in isopropyl acetate, 0.036 mL ethyl hexanoic acid, 18 hours reaction time. The results are shown in Table H:

TABLE H
Catalyst Screening (temperature:
100° C., pressure: 80 bar, time: 18 hours)

| | | Furan 39 | THF Int | Impurity | Teramep | Ring hydrog |
|---|---|---|---|---|---|---|
| | Catalyst* | (%) | 40 (%) | 48 (%) | 1 (%) | Byprod (%) |
| 49 | B103032-5 | 48.6 | 51.4 | 0.0 | 0.0 | 0 |
| 50 | 5% Pd/Silica- | 4.8 | 88.7 | 0.0 | 0.0 | 6.5 |
| | Alumina | | | | | |
| 51 | A570129-10 | 8.0 | 67.6 | 2.3 | 10.7 | 11.4 |
| 52 | A501023-10 | 3.5 | 72.5 | 1.5 | 9.9 | 12.6 |
| 54 | A470036-10 | 5.8 | 67.5 | 3.2 | 12.1 | 11.4 |
| 55 | A470201-10 | 1.3 | 45.0 | 11.3 | 35.4 | 6.9 |
| 56 | E101 NE/W GG | 2.5 | 29.0 | 17.7 | 39.0 | 11.7 |

*Note:
E101 NE/W GG is a 10% Pd/C catalyst obtained from Sigma-Aldrich Inc. The rest catalysts are also 10% Pd/C catalysts and were obtained from Johnson Matthey Inc. Although they are all 10% Pd/C catalysts, different catalysts have different supporting materials regarding to the particle size, surface area, water percentage, etc.

The experiments in table H were run at 80 bar and showed poor conversion in most cases but the E101 NE/W GG gave a better conversion in this case. Ring hydrogenation byproducts were observed in a significant quantities.

Further experiments were conducted regarding different catalysts in Scheme 21. The following reaction conditions were used: 330 mg substrate, 24 mg dry weight catalyst, 30% IPA in isopropyl acetate, 0.036 mL ethyl hexanoic acid, 16 hours, 30 min. reaction time, not 18 hours. The results are shown in Table I:

TABLE I
Catalyst Screening (temperature:
100° C., pressure: 90 bar, time: 18 hours)

| | | Furan 39 | THF Int | Impurity | Teramep | Ring hydrog |
|---|---|---|---|---|---|---|
| | Catalyst* | (%) | 40 (%) | 48 (%) | 1 (%) | Byprod (%) |
| 73 | A501032-10 | 23.1 | 53.0 | 0.3 | 3.2 | 20.4 |
| 74 | A402028-10 | 3.2 | 59.8 | 7.8 | 22.6 | 6.7 |
| 75 | B103018-5 | 58.3 | 39.3 | 0.3 | 1.4 | 0.6 |
| 76 | 10R39 | 0.0 | 40.9 | 17.4 | 38.2 | 3.5 |
| 78 | 10R490 | 0.2 | 88.2 | 2.0 | 6.3 | 3.4 |
| 79 | A402032-10 | 0.9 | 43.5 | 7.7 | 36.4 | 11.5 |
| 80 | E101 MLP | 0.0 | 26.0 | 21.0 | 48.8 | 4.2 |

*Note:
E101 MLP is a 10% Pd/C catalyst obtained from Sigma-Aldrich Inc. The rest catalysts are also 10% Pd/C catalysts and were obtained from Johnson Matthey Inc. Although they are all 10% Pd/C catalysts, different catalysts have different supporting materials regarding to the particle size, surface area, water percentage, etc.

Table I shows evaluations of some alternative catalysts under standard conditions. The A402032-10 catalyst showed reasonable selectivity of terameprocol 1 over impurity 48 but it also generated a significant amount of over-hydrogenation products even though conversion was only moderate.

Further experiments were conducted regarding different catalysts in Scheme 21, but at a higher temperature of 120° C. The following other reaction conditions were used: 330 mg substrate, 2.5 mol % Pd, 30% IPA in isopropyl acetate, 0.036 mL ethyl hexanoic acid, 18 hours reaction time. The results are shown in Table J:

TABLE J
Catalyst Screening (temperature:
120° C., pressure: 90 bar, time: 18 hours)

| | | Furan 39 | THF Int | Impurity | Teramep | Ring hydrog |
|---|---|---|---|---|---|---|
| | Catalyst* | (%) | 40 (%) | 48 (%) | 1 (%) | Byprod (%) |
| 81 | 10R37 | 0 | 88.5 | 0.3 | 1.6 | 9.6 |
| 82 | 20R91 | 0 | 0.0 | 35.8 | 7.3 | 56.9 |
| 83 | 10R394 | 0 | 7.9 | 40.4 | 38.7 | 13.1 |
| 84 | A470201-10 | 0 | 9.0 | 26.7 | 40.9 | 23.0 |
| 86 | E101 NE/W GG | 0 | 0.1 | 33.0 | 17.3 | 49.6 |
| 87 | A402032-10 | 0 | 1.0 | 25.2 | 33.1 | 40.7 |
| 88 | 10R39 | 0 | 1.2 | 44.6 | 37.7 | 16.6 |

*Note:
E101 NE/W GG is a 10% Pd/C catalyst obtained from Sigma-Aldrich Inc. The rest catalysts are also 10% Pd/C catalysts and were obtained from Johnson Matthey Inc. Although they are all 10% Pd/C catalysts, different catalysts have different supporting materials regarding to the particle size, surface area, water percentage, etc.

In table J it can be clearly seen that the elevated temperature gave much more ring hydrogenation. The inventor realized that this will be a function of reaction time as the products continue to hydrogenate once formed, so if the reaction were stopped at an earlier point, the selectivity to non-ring hydrogenated products would be better.

A combination of different catalysts and solvents were tested in Scheme 21. The following reaction conditions were used for all reactions: 330 mg substrate, 4 mol % Pd, 0.036 mL ethyl hexanoic acid, 100° C. and 90 bar H₂, 18 hours reaction time. The results are shown in Table K.

TABLE K
Solvent Screening (catalyst loading: 4 mol % Pd; temperature: 100° C.,
pressure: 90 bar, time: 18 hours)

| | | | Furan | THF Int 40 | Impurity 48 | Teramep | Ring hydrog |
|---|---|---|---|---|---|---|---|
| | Catalyst* | Solvent | 39 (%) | (%) | (%) | 1 (%) | Byprod (%) |
| 91 | A402032-10 | 2-propanol | 0 | 0 | 19.0 | 0.0 | 81.0 |
| 92 | A402032-10 | 2-butanol | 0 | 0 | 28.0 | 11.6 | 60.3 |
| 94 | 10R90 | 30% IPA/IP | 0 | 0 | 73.4 | 13.8 | 12.8 |
| | | acetate | | | | | |
| 95 | E101 NE/W GG | 30% IPA/IP | 0 | 41.8 | 14.7 | 34.9 | 8.1 |
| | | acetate | | | | | |
| 96 | A402032-10 | 30% IPA/IP | 0 | 2.5 | 24.8 | 52.9 | 19.9 |
| | | acetate | | | | | |

*Note:
E101 NE/W GG is a 10% Pd/C catalyst obtained from Sigma-Aldrich Inc. The rest catalysts are also 10% Pd/C catalysts and were obtained from Johnson Matthey Inc. Although they are all 10% Pd/C catalysts, different catalysts have different supporting materials regarding to the particle size, surface area, water percentage, etc.

Further experiments were conducted regarding different solvents and certain different catalysts in Scheme 21. The following conditions were used for all reactions: 330 mg substrate, 3 mol % Pd, 5 mL solvent, no acid, 100° C., 90 bar H₂, 18 hours reaction time. The results are shown in Table L:

TABLE L
Solvent Screening (catalyst loading: 3 mol % Pd; temperature:
100° C., pressure: 90 bar, time: 18 hours)

| | | | Furan | THF Int | Impurity | Teramep | Ring hydrog |
|---|---|---|---|---|---|---|---|
| | Catalyst* | Solvent | 39 (%) | 40 (%) | 48 (%) | 1 (%) | Byprod (%) |
| 98 | 10R39 | Chloroform | | Stirrer | failed | | |
| 99 | 10R39 | n-Butyl acetate | 0.0 | 2.3 | 31.6 | 62.8 | 3.4 |
| 100 | 10R39 | 2-butanol | 0.0 | 0.0 | 57.5 | 32.9 | 9.6 |
| 102 | A402032-10 | Chloroform | 24.6 | 6.6 | 22.8 | 12.3 | 33.7 |
| 103 | A402032-10 | n-Butyl acetate | 0.0 | 8.2 | 31.9 | 54.8 | 5.1 |
| 104 | A402032-10 | 2-butanol | | Leak | | | |

*Note:
All catalysts are 10% Pd/C catalysts and were obtained from Johnson Matthey Inc. Although they are all 10% Pd/C catalysts, different catalysts have different supporting materials regarding to the particle size, surface area, water percentage, etc.

Tables K and L show the effect of catalyst loading and solvent variations. At 4 mol % Pd loading (Table K) the reaction clearly goes too far in most cases and ring hydrogenation products are formed in large quantities. When loading was cut to 3 mol %, and the acid was excluded, the reaction in butyl acetate seemed to give reasonable selectivity (Table L). It is apparent from the results that the use of a single, polar, protic solvent gave fast reaction but poor selectivity. Non-protic solvents, whether polar or apolar, gave slower conversion of the THF intermediate 40 into products and over hydrogenated products.

The results in Table L show the comparison of the use of isopropyl acetate versus n-butyl acetate. It can be seen that for both catalysts 10R39 and A402032-10 the use of n-butyl acetate gave vastly superior results.

Further experiments were conducted regarding different solvents and certain different catalysts in Scheme 21. The following conditions were used for all reactions: 330 mg substrate, 3 mol % Pd, 5 mL solvent, 100° C., 90 bar H₂, 18 hours reaction time. The results are shown in Tables M and N:

TABLE M
Solvent Screening (catalyst loading: 3 mol % Pd; temperature
100° C., pressure: 90 bar, time 18 hours)

| | | | Furan | THF Int | Impurity | Teramep | Ring hydrog |
|---|---|---|---|---|---|---|---|
| | Catalyst* | Solvent | 39 (%) | 40 (%) | 48 (%) | 1 (%) | Byprod (%) |
| 105 | 10R39 LS0369 | Isopropyl acetate | 0.0 | 58.2 | 14.0 | 25.4 | 2.4 |
| 106 | A-402032-10 | n-Butyl acetate | 0.0 | 8.4 | 30.0 | 57.6 | 4.0 |
| 107 | 10R37 | Isopropyl acetate | 7.0 | 87.8 | 1.0 | 2.0 | 2.3 |
| 108 | 10R394 | Isopropyl acetate | 0.0 | 10.7 | 30.6 | 53.7 | 5.1 |
| 111 | E101 NE/W GG | Isopropyl acetate | 0.8 | 75.6 | 6.9 | 14.1 | 2.6 |
| 112 | A-402032-10 | Isopropyl acetate | 0.0 | 78.9 | 4.3 | 14.5 | 2.3 |

*Note:
E101 NE/W GG is a 10% Pd/C catalyst obtained from Sigma-Aldrich Inc. The rest catalysts are also 10% Pd/C catalysts and were obtained from Johnson Matthey Inc. Although they are all 10% Pd/C catalysts, different catalysts have different supporting materials regarding to the particle size, surface area, water percentage, etc.

TABLE N
Solvent Screening (catalyst loading: 3 mol % Pd; temperature:
100° C., pressure: 90 bar, time: 18 hours)

| | | | Furan | THF Int | Impurity | Teramep | Ring hydrog |
|---|---|---|---|---|---|---|---|
| | Catalyst* | Solvent | 39 (%) | 40 (%) | 48 (%) | 1 (%) | Byprod (%) |
| 113 | A-402032-10 | n-Butyl acetate | 0.0 | 87.8 | 1.5 | 8.6 | 2.1 |
| 114 | 10R39 | n-Butyl acetate | 0.0 | 15.4 | 27.1 | 54.0 | 3.5 |
| 115 | E101 NE/W GG | n-Butyl acetate | 0.4 | 90.4 | 2.2 | 5.3 | 1.7 |
| 116 | A-402032-10 | n-Butyl acetate | 0.0 | 53.1 | 7.7 | 22.9 | 15.3 |
| 118 | 10R39 | n-Butyl acetate | 0.0 | 32.7 | 26.0 | 36.2 | 5.2 |
| 119 | A-402032-10 | 10% 2- | 0.0 | 74.0 | 4.6 | 13.7 | 7.7 |
| | | Butanol/n-Butyl | | | | | |
| | | acetate | | | | | |
| 120 | E101 NE/W GG | n-Butyl acetate | 0.5 | 93.5 | 0.6 | 3.1 | 2.3 |

*Note:
E101 NE/W GG is a 10% Pd/C catalyst obtained from Sigma-Aldrich Inc. The rest catalysts are also 10% Pd/C catalysts and were obtained from Johnson Matthey Inc. Although they are all 10% Pd/C catalysts, different catalysts have different supporting materials regarding to the particle size, surface area, water percentage, etc.

Using either isopropyl or n-butyl acetates (Tables M and N) as single solvents appeared to give lower amounts of ring hydrogenation products while giving a maximum selectivity of 2:1 for terameprocol over impurity 48.

Further experiments were conducted regarding different catalysts in Scheme 21. The following reaction conditions were used for all reactions: 330 mg substrate, 3 mol % Pd, 5 mL solvent, 100° C., 90 bar H₂, 16 hours reaction time. The results are shown in Tables O and P:

TABLE O
Catalyst Screening (catalyst loading: 3 mol % Pd; temperature:
100° C., pressure: 90 bar, time: 18 hours)

| | | | Furan | THF Int | Impurity | Teramep | Ring hydrog |
|---|---|---|---|---|---|---|---|
| | Catalyst* | Solvent | 39 (%) | 40 (%) | 48 (%) | 1 (%) | Byprod (%) |
| 121 | 10R39 | n-Butyl acetate | 0.0 | 76.6 | 6.4 | 15.3 | 1.7 |
| 122 | 10R39# | n-Butyl acetate | 0.0 | 78.7 | 6.9 | 12.6 | 1.8 |
| 123 | E101 NE/W GG | n-Butyl acetate | 1.5 | 82.4 | 3.9 | 9.7 | 2.5 |
| 124 | E101 NE/W GG# | n-Butyl acetate | 0.5 | 85.7 | 3.6 | 8.5 | 1.7 |
| 126 | A402032-10 | n-Butyl acetate | 0.0 | 24.0 | 20.0 | 49.0 | 7.0 |

*Note:
E101 NE/W GG is a 10% Pd/C catalyst obtained from Sigma-Aldrich Inc. The rest catalysts are also 10% Pd/C catalysts and were obtained from Johnson Matthey Inc. Although they are all 10% Pd/C catalysts, different catalysts have different supporting materials regarding to the particle size, surface area, water percentage, etc.
#catalyst charge added in 2 stages at 0 and 3 hours

TABLE P
Catalyst Screening (catalyst loading: 3 mol % Pd; temperature:
100° C., pressure: 90 bar, time: 18 hours)

| | | | Furan | THF Int | Impurity | Teramep | Ring hydrog |
|---|---|---|---|---|---|---|---|
| | Catalyst* | Solvent | 39 (%) | 40 (%) | 48 (%) | 1 (%) | Byprod (%) |
| 129 | E101 NE/W GG. | n-Butyl acetate | 3.1 | 89.4 | 1.2 | 3.6 | 2.7 |
| 130 | 10R39 | n-Butyl acetate | 0.0 | 46.7 | 13.9 | 33.9 | 5.8 |
| 136 | A402032-10 | n-Butyl acetate | 1.1 | 88.9 | 0.9 | 5.5 | 3.6 |

*Note:
E101 NE/W GG is a 10% Pd/C catalyst obtained from Sigma-Aldrich Inc. The rest catalysts are also 10% Pd/C catalysts and were obtained from Johnson Matthey Inc. Although they are all 10% Pd/C catalysts, different catalysts have different supporting materials regarding to the particle size, surface area, water percentage, etc.

Further experiments were conducted regarding different catalysts and solvents in Scheme 21. The following reaction conditions were used for all reactions: 330 mg substrate, 3 mol % Pd, 5 mL solvent, 100° C., 90 bar H₂, 18 hours reaction time. The results are shown in Table Q:

TABLE Q
Catalyst and Solvent Screening (catalyst loading: 3 mol
% Pd; temperature: 100° C., 90 bar, time: 18 hours)

| | | | Furan | THF Int | Impurity | Teramep | Ring hydrog |
|---|---|---|---|---|---|---|---|
| | Catalyst* | Solvent | 39 (%) | 40 (%) | 48 (%) | 1 (%) | Byprod (%) |
| 137 | A402032-10 | n-Butyl acetate | 0.1 | 86.9 | 1.3 | 6.7 | 5.0 |
| 138 | A402032-10 | n-Butyl acetate | 0.1 | 84.3 | 3.9 | 9.7 | 2.0 |
| 139 | A402032-10 | Isopropyl acetate | 0.0 | 76.2 | 2.5 | 12.9 | 8.0 |
| 140 | A402032-10 | 50% 2-Butanol/n- | 0.0 | 38.4 | 7.9 | 36.1 | 17.6 |
| | | Butyl acetate | | | | | |
| 142 | 10R39 | n-Butyl acetate | 0.2 | 44.9 | 13.4 | 38.7 | 2.7 |
| 143 | 10R39 | Isopropyl acetate | 0.2 | 64.4 | 10.6 | 22.3 | 2.4 |
| 144 | 10R39* | n-Butyl acetate | 0.0 | 84.1 | 5.3 | 9.3 | 1.2 |

*Note:
The catalysts are 10% Pd/C catalysts, different catalysts have different supporting materials regarding to the particle size, surface area, water percentage, etc.

Further experiments were conducted regarding different catalysts and solvents in Scheme 21. The following conditions were used for all reactions: 330 mg substrate, 3 mol % Pd excepted those indicated, 5 mL solvent, 100° C., 90 bar H₂, 18 hours reaction time. The results are shown in Table R:

TABLE R
Catalyst and Solvent Comparisons

| | | | Furan | THF Int | Impurity | Teramep | Ring hydrog |
|---|---|---|---|---|---|---|---|
| | Catalyst* | Solvent | 39 (%) | 40 (%) | 48 (%) | 1 (%) | Byprod (%) |
| 145 | A402032-10** | n-Butyl acetate | 0.0 | 92.9 | 0.4 | 3.1 | 3.6 |
| 146 | A402032-10 | n-Butyl acetate | 8.5 | 84.7 | 0.1 | 1.1 | 5.6 |
| 147 | A402032-10*** | n-Butyl acetate | 0.9 | 62.5 | 3.9 | 18.9 | 13.8 |
| 148 | A402032-10** | Isopropyl acetate | 0.6 | 62.8 | 3.6 | 19.0 | 14.0 |
| 150 | 10R39 | n-Butyl acetate | 0.3 | 62.0 | 11.3 | 23.9 | 2.5 |
| 151 | 10R39 | Isopropyl acetate | 0.0 | 67.7 | 10.5 | 17.0 | 4.8 |

*Note:
The catalysts are 10% Pd/C catalysts and were obtained from Johnson Matthey Inc. Although they are all 10% Pd/C catalysts, different catalysts have different supporting materials regarding to the particle size, surface area, water percentage, etc.
**2.5 mol % Pd used.
***3.5 mol % Pd used.

Further experiments were conducted regarding certain catalysts, while varying solvent concentration in Scheme 21. The following reaction conditions were used for all reactions: 330 mg substrate, 3 mol % Pd, 100° C., 90 bar H₂, 18 hours reaction time. The results are shown in Table S:

TABLE S
Catalyst And Solvent Concentration Screening

| | | | Furan | THF Int | Impurity | Teramep | Ring hydrog |
|---|---|---|---|---|---|---|---|
| | Catalyst* | Solvent | 39 (%) | 40 (%) | 48 (%) | 1 (%) | Byprod (%) |
| 153 | 10R39 | 30% IPA/IP | 9.5 | 87.3 | 0.1 | 0.8 | 2.3 |
| | | acetate | | | | | |
| 154 | 10R39 | 30% IPA/IP | 0.6 | 28.3 | 18.5 | 47.4 | 5.3 |
| | | acetate | | | | | |
| 155 | A402032-10 | 30% IPA/IP | 0.1 | 46.2 | 7.1 | 31.2 | 15.5 |
| | | acetate | | | | | |
| 156 | E101 G.G | 30% IPA/IP | 0.0 | 27.0 | 23.1 | 40.2 | 9.8 |
| | | acetate | | | | | |
| 158 | A402032-10 | 40% IPA/IP | 0.0 | 14.3 | 21.0 | 44.2 | 20.5 |
| | | acetate | | | | | |
| 159 | A402032-10 | 50% IPA/IP | 0.0 | 0.7 | 28.5 | 29.3 | 41.5 |
| | | acetate | | | | | |
| 160 | A402032-10 | IPA | 0.0 | 6.5 | 21.1 | 54.6 | 17.5 |

*Note:
The catalysts are 10% Pd/C catalysts and were obtained from Johnson Matthey Inc. Although they are all 10% Pd/C catalysts, different catalysts have different supporting materials regarding to the particle size, surface area, water percentage, etc.

Further experiments were conducted regarding different catalysts and solvents in Scheme 21. The following reaction conditions were used for all reaction: 330 mg substrate, 3 mol % Pd, 5 mL solvent, 100° C., 90 bar H₂, 18 hours reaction time. The results are shown in Tables T and U:

TABLE T
Catalyst Screening

| | | | Furan | THF Int | Impurity | Teramep | Ring hydrog |
|---|---|---|---|---|---|---|---|
| | Catalyst* | Solvent | 39 (%) | 40 (%) | 48 (%) | 1 (%) | Byprod (%) |
| 161 | 10R39 | 30% IPA/IP | 0.0 | 0.0 | 40.5 | 36.9 | 22.6 |
| | | acetate | | | | | |
| 162 | 10R39** | 30% IPA/IP | 0.0 | 0.0 | 51.4 | 22.7 | 25.9 |
| | | acetate | | | | | |
| 163 | A402032-10 | 30% IPA/IP | 0.0 | 11.9 | 17.5 | 45.0 | 25.7 |
| | | acetate | | | | | |
| 164 | A402032-10* | 30% IPA/IP | 0.4 | 32.2 | 9.6 | 36.5 | 21.2 |
| | | acetate | | | | | |
| 166 | 10R39 | IPA | 0.9 | 57.6 | 4.5 | 17.5 | 19.4 |
| | M07048A | | | | | | |
| 167 | 10R39 | IPA | 0.0 | 0.0 | 41.1 | 38.7 | 20.1 |
| 168 | A402032-10 | IPA | 0.0 | 0.0 | 32.9 | 18.8 | 48.2 |

*Note:
The catalysts are 10% Pd/C catalysts and were obtained from Johnson Matthey Inc. Although they are all 10% Pd/C catalysts, different catalysts have different supporting materials regarding to the particle size, surface area, water percentage, etc.
**4 mol % Pd

TABLE U
Catalyst Screening

| | | | Furan | THF Int | Impurity | Teramep | Ring hydrog |
|---|---|---|---|---|---|---|---|
| | Catalyst* | Solvent | 39 (%) | 40 (%) | 48 (%) | 1 (%) | Byprod (%) |
| 169 | SCY-023 | 30% IPA/IP acetate | | LEAK | | | |
| | (161) | | | | | | |
| 170 | 10R39** | 30% IPA/IP acetate | 0.0 | 3.7 | 25.8 | 65.1 | 5.4 |
| 171 | SCY-023 | 30% IPA/IP acetate | 0.0 | 7.9 | 19.4 | 44.1 | 22.7 |
| | (163) | | | | | | |
| 172 | A-402032- | 30% IPA/IP acetate | 0.5 | 59.3 | 5.3 | 26.1 | 8.9 |
| | 10 | | | | | | |
| 174 | 10R39*** | 30% IPA/IP acetate | 0.0 | 9.2 | 24.7 | 60.4 | 5.6 |
| 175 | 10R39 | IPA | 0.0 | 4.1 | 27.8 | 51.9 | 16.2 |
| 176 | 10R39 | 30% IPA/IP acetate + | 0.0 | 51.2 | 25.4 | 22.5 | 0.9 |
| | | 10% Formic Acid | | | | | |

*Note:
The catalysts are 10% Pd/C catalysts and were obtained from Johnson Matthey Inc. Although they are all 10% Pd/C catalysts, different catalysts have different supporting materials regarding to the particle size, surface area, water percentage, etc.
**4 mol % Pd
***6 mol % Pd

Studies were done to determine the effect of different amounts of the Pd catalyst in the reaction of Scheme 21. The following reaction conditions were used: 200 mg substrate, 100° C. and 90 bar H₂, 3 hours reaction time. The results are shown in Table V:

TABLE V
Catalyst Loading Trials

| | | Mol % | | Furan | THF Int | Impurity | Teramep | Ring hydrog |
|---|---|---|---|---|---|---|---|---|
| | Catalyst* | Pd | Solvent | 39 (%) | 40 (%) | 48 (%) | 1 (%) | Byprod (%) |
| 177 | 10R39 | 5 | 30% IPA/IP | 0.0 | 17.5 | 20.3 | 59.5 | 2.1 |
| | LR0434 | | acetate | | | | | |
| 178 | 10R39 | 7 | 30% IPA/IP | 0.0 | 6.7 | 24.1 | 55.8 | 7.9 |
| | | | acetate | | | | | |
| 179 | 10R39 | 8.8 | 30% IPA/IP | 0.0 | 0.0 | 26.8 | 46.9 | 13.8 |
| | | | acetate | | | | | |
| 180 | 10R39 | 10.2 | 30% IPA/IP | 0.0 | 1.1 | 27.3 | 61.6 | 3.4 |
| | | | acetate | | | | | |
| 182 | A402032-10 | 3.7 | 30% IPA/IP | 0.0 | 61.8 | 3.0 | 13.7 | 6.3 |
| | | | acetate | | | | | |
| 183 | A402032-10 | 5.3 | 30% IPA/IP | 0.0 | 54.3 | 5.6 | 26.4 | 12.8 |
| | | | acetate | | | | | |
| 184 | A402032-10 | 7.2 | 30% IPA/IP | 0.0 | 84.4 | 1.3 | 7.8 | 5.5 |
| | | | acetate | | | | | |

*Note:
The catalysts are 10% Pd/C catalysts and were obtained from Johnson Matthey Inc. Although they are all 10% Pd/C catalysts, different catalysts have different supporting materials regarding to the particle size, surface area, water percentage, etc.

The results included in Tables M-R seem to suffer from poor conversion compared to previous experiments. Ring hydrogenation byproducts were always higher with the A402032-10 catalyst than with the 10R39 catalyst. Experiments in Table S were conducted with additional IPA to increase activity. Conversions were much improved for the two catalysts but the amount of over-hydrogenated products were considerably higher. The use of a powder catalyst, rather than a water-wet paste, showed much lower activity. The results from Table T indicate that an increase in catalyst loading is not particularly beneficial under these conditions. In all cases, over-hydrogenated byproducts were seen to be high but in these cases the major byproduct was the over-hydrogenation of the desired product. Two trials were then conducted using shorter time periods. Table U shows results from 3 hours of reaction and it can be clearly seen that using the 10R39 catalyst still achieves very high conversion to products and the reaction is cleaner-low ring hydrogenation products. Table V shows no significant benefit from higher catalyst loadings under these conditions.

Clearly, the results from these experiments support activity of the 10R39 catalyst for the second general step conversion. By using this catalyst and limiting the reaction time one can achieve near quantitative conversion to impurity 48 and terameprocol lwith low content of ring hydrogenation byproducts. The selectively to terameprocol still appears to be only moderate.

The results from the studies indicate that the conversion of the furan 39 to the desired terameprocol product is difficult to achieve selectively because of the competing reactions: ring-closing to form the impurity 48 or ring hydrogenation. The ring hydrogenation can be minimized by optimization of reaction time, catalyst loading, catalyst type and solvent choice. However, under the various conditions assessed, the ring-closing side reaction is very difficult to stop. Both chemistries are faster when polar, protic solvents are used, but these conditions tend to favor formation of the byproduct 48. Polar, aprotic solvents such as the acetates (n-butyl or isopropyl) seem to be the best compromise for activity and selectivity, with added IPA to increase activity. The use of added acidity does not seem necessary when the 10R39 catalyst is employed.

Based on small scale (i.e. 200 mg or 300 mg compound 39) screening experiments as shown in Tables B˜V, a preferred reaction conditions for the direct conversion of the furan molecular 39 to terameprocol 1 are the following: (a) temperature: 80-120° C.; (b) reaction pressure: 800 psi ˜1310 psi; (c) reaction time: 2 h ˜24 h, more preferable 15 h ˜18 h; (d) catalyst loading: 2.5 mol %˜4.5 mol % Pd; and preferable catalysts are 10% Pd/C catalyst, more specifically, 10R39 and E101 NE/W GG are better among all catalysts that the present investor has tested, (e) preferable solvents are isopropyl acetate and isopropanol, preferable ratio of isopropyl acetate (IPA):isopropanol (IP) is 2:8˜4:6 (V:V).

### 4. Scale-Up of Conversion of Furan Compound 39 to Terameprocol 1

In preparation for scaling up the hydrogenation of furan 39, it was found that the reaction stalled after 18 hours, giving a 1:1 mixture of terameprocol: THF intermediate 40. To solve the problem of reproducibility, additional development work was performed.

The role of solvent was examined further while keeping the other parameters constant (i.e., 4 mol % 10R39 catalyst, 100° C., 1300 psi H₂). Based on the findings from the catalyst-screening work that a wet catalyst performed better than a dry catalyst, the alcohol component, which is known to both speed the desired reaction and generate more cyclized impurity, was originally replaced with 10% water. Due to trans-etherification issues observed with isopropyl acetate, it was replaced with the higher-boiling solvent n-propyl acetate (bp 100° C.). Interestingly, the presence of water prevented the reaction from stalling and appeared to perform well within the first 3 hours of the reaction. However, over longer time periods, a 1:1 mixture of terameprocol and a byproduct, having the same mass as terameprocol by LC/MS, was formed. Upon work-up, the reaction mixture smelled strongly of acetic acid, and the aqueous washes were found to have pH 3-4. This suggested that under the reaction conditions the n-propyl acetate underwent hydrolysis to acetic acid, which could potentially catalyze the hydrolysis of furan 39 to racemic diketone (rac-diketone) 38 (Scheme 22). Under the reaction conditions rac-diketone 38 could undergo hydrogenation to both terameprocol 1 and rac-terameprocol 1a.

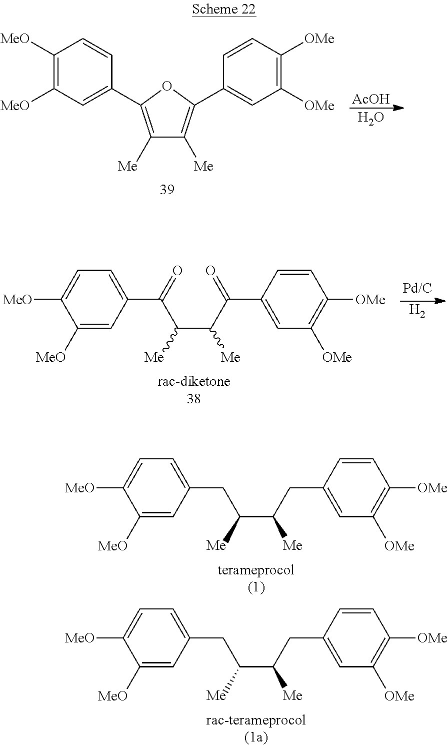

To avoid the possibility of solvent hydrolysis, 10% water in methylcyclohexane was examined. Under the reaction conditions, formation of rac-terameprocol 1a, as well as over-hydrogenated products, were observed by LC/MS and nuclear magnetic resonance (NMR), respectively. Therefore, a different strategy to avoid furan hydrolysis was attempted by first converting furan 39 to THF intermediate 40 in n-propyl acetate prior to adding water. When 10R39 catalyst was used at 1000 psi H₂ and 100° C. in n-propyl acetate, furan 39 converted slowly to the THF intermediate 40 (26% by LC/MS). Interestingly, conversion to terameprocol (about 31%) and cyclized impurity 48 (12%) was observed along with some unreacted starting material (about 31%). No over-hydrogenated products appeared to form. The reaction stalled, however, after complete conversion of furan 39, with 27% THF intermediate 40 remaining.

The following observations led to important conclusions regarding the 10R39 catalyst (a type of 10% Pd/C catalyst, available from Johnson Matthey Inc.). First, pressures higher than 1000 psi were not necessary to convert THF intermediate 40 to terameprocol. In fact, running the hydrogenation at lower pressure minimized or eliminated over-hydrogenation. Second, although the 10R39 catalyst had less difficulty in the hydrogenolysis of THF intermediate 40 to terameprocol 1, it was less effective at the initial hydrogenation of furan 39. In comparison, the E101 NE/W GG catalyst (a type of 10% Pd catalyst, available from Sigma-Aldrich) rapidly converted furan 39 to THF intermediate 40, but struggled to convert the THF intermediate to products at 1000 psi. Based on these conclusions a two-catalyst system was explored.

To receive the full benefit of the 10R39 catalyst, a mixture of E101 NE/W GG catalyst (0.5 mol %) and 10R39 catalyst (2 mol %) was used at 1000 psi H₂ in n-propyl acetate at 100° C. In theory, the E101 catalyst would readily hydrogenate furan 39 to THF intermediate 40. Afterwards, the 10R39 catalyst would easily mediate the hydrogenolysis of THF intermediate 40 to terameprocol. To prevent the reaction from stalling without risking furan hydrolysis, water could be added after all furan 39 converted to THF intermediate 40.

In practice, this approach was successful. A mixture of 10R39 catalyst (2 mol %) and E101 NE/W GG catalyst (0.5 mol %) in n-propyl acetate was pre-hydrogenated for 30 min. at room temperature. After adding furan 39 (40 g), the mixture was vigorously stirred under 1000 psi H₂ and 100° C. After 3-4 hours, the starting material was completely consumed and a mixture of THF intermediate 40 and products were obtained. The amount of THF intermediate 40 appeared to level at 15% and water (10% v/v) was added to drive the reaction to completion. After holding the mixture overnight, the mixture was filtered through Celite® filter material and washed with water, aqueous potassium carbonate solution (to remove residual acids) and brine. The product mixture was concentrated and then solvent-swapped into heptane. Terameprocol was crystallized from heptane, filtered and washed with additional heptane to give a 45% isolated yield with 98.5% purity (HPLC method).

### Scale-Up

The following specific, non-limiting examples of scale-up processes were performed according to the present invention, following Schemes 23 and 24 as described below.

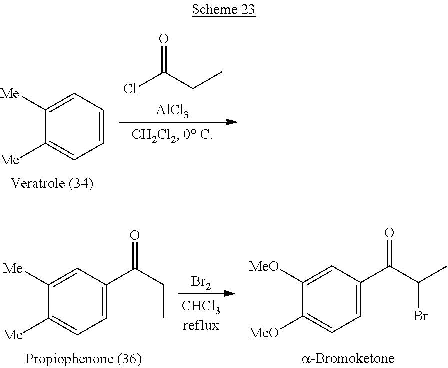

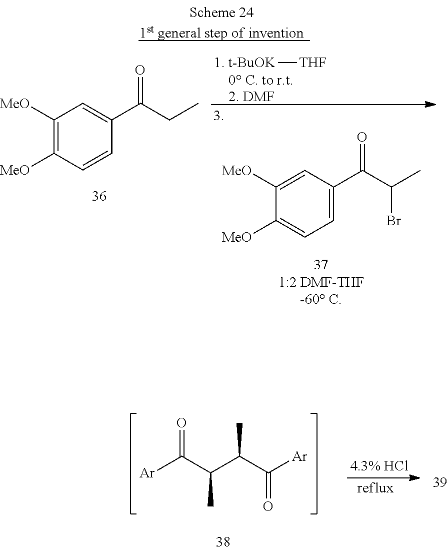

For each of the Examples, NMR data were acquired using a Varian 400 MHz spectrometer. LC and MS data were acquired using a Thermo-Finnegin Surveyor HPLC equipped with a Phenomenex C18 5® column connected to an AQA mass spectrometer. Mobile phase A: water+1% CAN+1% formic acid). Mobile phase B: MeOH. Gradient LC method: 5% mobile phase B to 100% B over 5 min. Elemental analysis was performed by an independent laboratory.

### Example 1

### Preparation of 3,4-Dimethoxypropiphenone 36

This compound was prepared by a modified procedure based on the method of Perry et al. (1972) supra, as outlined in Scheme 23.

To a 5 L, 4-necked round bottom flask (equipped with N₂ inlet, overhead magnetic stir drive, addition funnel, thermocouple, and condenser) was added aluminum chloride (349 g, 2.61 mol) followed by CH₂Cl₂ (870 mL). The suspension was cooled to −10° C. using a dry ice/acetone bath. Propionyl chloride (138 g, 1.49 mol) in CH₂Cl₂ (145 mL) was added in portions via addition funnel over 15 min. keeping the mixture between −2° C. and 2° C. The mixture was stirred for an additional 10 min. Veratrole 34 (170 g, 1.23 mol) in CH₂Cl₂ (100 mL) was added via the addition funnel over 20 min. while keeping the reaction mixture between −4° C. and 1° C. After 5 min., TLC showed complete consumption of starting material (SiO₂, 1:1 EtOAc-heptane, UV, veratrole Rf=0.54, propiophenone Rf=0.42). The reaction was cooled to −10° C. and aqueous 3N HCl (2 L) was slowly and cautiously added over 25 min. while keeping the reaction mixture between −1° C. and 16° C. The phases were separated and the aqueous phase was extracted once with CH₂Cl₂ (500 mL). The combined CH₂Cl₂ extract was washed with 3N NaOH (1 L), dried over MgSO₄ (34.5 g), then concentrated in vacuo to give a viscous oil. The oil dissolved in hot MeOH (300 mL) and the solution was held at 0-5° C. for 16 hours. The resulting white solids were broken up with spatula and vacuum filtered. The filter cake was washed with heptane (125 mL) and dried on the funnel (179.9 g). A second crop of solids was obtained by concentrating the mother liquor, diluting with MeOH and holding at 0-5° C. for 3 hours (15 g). The crop 1 and 2 solids were combined and dried in a vacuum oven (30° C., 18 hours) to give propiophenone 36 as a white solid (192 g, 80.5% yield, purity>98% as determined by HPLC). ¹H NMR (CDCl₃, 400 MHz) δ 1.21 (t, 3H, J=7.3 Hz), 2.96 (q, 2H, J=7.3 Hz), 3.93 (s, 3H), 3.94 (s, 3H), 6.88 (d, 1H, J=8.3 Hz), 7.54 (d, 1H, J=1.6 Hz), 7.58 (dd, 1H, J=8.3, 1.6 Hz). ¹³C NMR (CDCl₃, 100 MHz) δ 8.27, 30.98, 55.64, 55.73, 109.74, 109.83, 122.24, 129.87, 148.69, 152.79, 199.10. LCMS (m/z=194.8).

### Example 2

### Preparation of 2-Bromo-3,4-Dimethoxypropiophenone 37

This compound was prepared by a modified procedure based on the method of Perry et al. (1972), supra, as outlined in Scheme 23.

A 3 L, 3-neck round bottom flask was fitted with an additional funnel, overhead magnetic stir, thermocouple, N₂ inlet and condenser. The condenser was vented into a base trap containing NaOH (50.2 g, 1.26 mol) in 1.8 L deionized water. The vessel was charged with propiophenone 36 (241.55 g, 1.25 mol) and chloroform (900 mL). The mixture was heated to 62-64° C. To the refluxing solution was added a solution of bromine (203.9 g, 1.27 mol, in 300 mL chloroform) via the addition funnel over 35 min. while vigorously stirring during addition. After addition, the mixture was vigorously stirred for 20 min. and cooled at 20° C. The solvent was removed in vacuo and the resulting solids were dissolved in CHCl₃ (250 mL) and MeOH (625 mL). The solution was concentrated until solids formed and the slurry was cooled to 0-5° C. hand held for 10 min. The slurry was vacuum filtered on a 2 L fritted funnel (medium frit), and the filter cake was washed with cold MeOH (2×50 mL). The solids were dried in a vacuum oven (35° C., 15 hours) to give 228 g α-bromoketone 37 (67% yield). A second crop was obtained by concentrating the mother liquor to a solid, and crystallizing from hot MeOH (300 mL) to give an additional 58 g α-bromoketone 37 (17.3% w/w yield). ¹H NMR (CDCl₃, 400 MHZ) δ 1.90 (d, 3H, J=6.7 Hz), 3.95 (s, 3H), 3.96 (s, 3H), 5.29 (q, 1H, J=6.7 Hz), 6.91 (d, 1H, J=8.4 Hz), 7.59 (d, 1H, J=2.0 Hz), 7.66 (dd, 1H, J=8.4, 2.0 Hz). ¹³C NMR (CDCl₃, 100 MHz) δ 20.23, 41.13, 55.90, 109.96, 111.03, 123.35, 126.85, 149.10, 153.69, 191.94. LC/MS (m/z=274.8).

### First General Step of Invention

### Example 3

### Preparation of 3,4-Dimethyl-2,5-bis(3,4-Dimethoxyphenyl)furan 39

As outlined in Scheme 23, to a dry 5 L, 3-necked round bottom flask (equipped with an overhead magnetic stir drive, addition funnel, thermocouple and N₂ inlet and outlet) was added solid 97% t-BuOK (103 g, 898 5 mmol, corrected for purity), followed by THF (615 mL). The solution was cooled to 0-1° C. with an ice-water bath. A solution of propiophenone 36 (170 g, 876.3 mmol) in THF (340 mL) was added in portions over 15 min. while keeping the internal temperature<7° C. giving a white/yellow-white slurry. After 15 min. the mixture was warmed to 18° C. and DMF (850 mL) was added via the addition funnel over 2 min. giving a clear yellow/orange solution. After 15 min, the reaction mixture was cooled to −70° C. using a dry ice/acetone bath. With vigorous stirring, a solution of α-bromoketone 37 (240 g, 876.3 mmol) in 2:1 THF-DMF (510 mL) was added in portions over 25 min. while maintaining an internal temperature between −60° C. and −55° C. After an additional 15 min. at −60° C., the reaction was complete as determined by LC/MS analysis. The reaction was quenched at −60° C. with water (900 mL) containing 70 mL 1N HCl and the reaction was warmed to 18-20° C. over 1 hour. The bulk of THF was removed in vacuo (1415 mL solvent removed) and the resulting mixture was extracted with CH₂Cl₂ (1.5 L). The organic layer was separated and the aqueous layer (pH 2-3) was back extracted twice with CH₂Cl₂ (2×400 mL). The combined CH₂Cl₂ was washed with water (425 mL). The bulk of the CH₂Cl₂ (1550 mL) was removed in vacuo and was transferred to a 3-neck round bottom flask equipped with overhead magnetic stir, addition funnel and condenser. The resulting solution was heated to reflux (44° C.) and a solution of 3% HCl in MeOH (1.1 L) was added in a steady stream. Solids precipitated within 15-20 min. Reflux was continued (57° C.) for 1 hour and the mixture was cooled to 0-2° C. over 2 hours. The solids were vacuum filtered and the filter cake was washed with MeOH (400 mL), then heptane (400 mL). The white solids were dried in a vacuum oven (20 hours, 50° C.), giving 296 g of furan 39 (91.9% yield, purity>96.3% as determined by HPLC). ¹H NMR (CDCl₃, 400 MHz) δ 2.22 (s, 6H), 3.92 (s, 6H), 3.95 (s, 6H), 6.94 (d, 2H, J=6.9 Hz), 7.24-719 (m, 4H). ¹³C NMR (CDCl₃, 100 MHz) δ 9.82, 55.87, 55.90, 109.14, 111.25, 117.77, 118.36, 125.06, 146.86, 148.00, 148.92. LC/MS (m/z=368.8).

### Example 3A

### Preparation of 3,4-Dimethyl-2,5-bis(3,4-Dimethoxyphenyl)furan 39 at kilogram scale

The reaction scheme of this example is shown in Scheme 23, to a dry 50 L, 3-necked round bottom flask (equipped with a mechanical stirrer, addition funnel, thermocouple and N₂ inlet and outlet) was added solid 97% t-BuOK (774.8 g, 6.56 mol, corrected for purity), followed by THF (4.60 L). The solution was cooled to 0-4° C. with an ice water bath. A solution of propiophenone 36 (1.243 kg, 6.40 mol) in THF (2.50 L) was added in portions over 60 min. while keeping the internal temperature<7° C. giving a white/yellow-white slurry. After 15 min. the mixture was warmed to 10° C. and DMF (6.20 L) was added via the addition funnel over 15 min. giving a clear yellow/orange solution. After 15 min, the reaction mixture was cooled to −70° C. using a dry ice/acetone bath. With vigorous stirring, a solution of α-bromoketone 37 (1.748 kg, 6.4 mol) in a solution of 2:1 THF-DMF (THF: 2.5 L; DMF: 1.25 L) was added in portions over 90 min. while maintaining an internal temperature between −60° C. and −55° C. After an additional 15 min. at −60° C., the reaction was complete as determined by LC/MS analysis. The reaction was quenched at −60° C. with water (8.60 L) containing 1M HCl 510 mL and the reaction was warmed to 18-20° C. over 1 hour. The bulk of THF was removed in vacuo (9000 mL solvent removed) and the resulting mixture was extracted with CH₂Cl₂ (5.6 L). The organic layer was separated and the aqueous layer (pH 2-3) was back extracted twice with CH₂Cl₂ (2×7.6 L). The combined CH₂Cl₂ was washed with water (3.5 L). The bulk of the CH₂Cl₂ (8000 mL) was removed in vacuo and was transferred to a 3-neck round bottom flask equipped with mechanical stirrer, addition funnel and condenser. The resulting solution was heated to reflux (44° C.) and a solution of 3% HCl in MeOH (prepared by adding 460 mL acetyl chloride to 8000 mL methanol) was added in a steady stream over a period of 90 min. Solids precipitated within 15-20 min. Reflux was continued (54° C.) for 5 hours and the mixture was cooled to 0-2° C. over 2 hours. The solids were vacuum filtered and the filter cake was washed with MeOH (2920 mL), then heptane (2920 mL). The white solids were dried in a vacuum oven (20 hours, 50° C.), then the solid was stirred and crushed to break down larger pieces. The solid was dried in a vacuum oven (20 hours, 50° C.). The procedure was repeated 3 times till solid was completely dried (no weight loss between drying turns), which gave 2194 g of furan 39 (91% yield, purity>98% as determined by HPLC). Analytical data was identical to example 3.

### Second General Step of Invention—Preparation of Terameprocol

In addition to Scheme 24, the second general step is

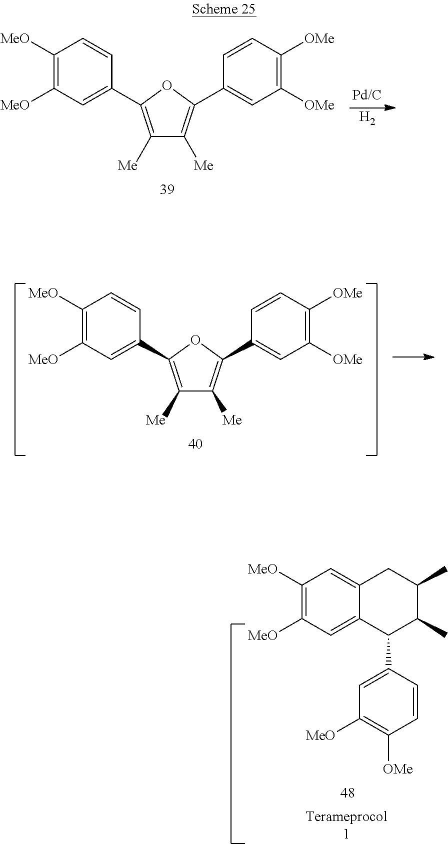

### Example 4

### Preparation of Terameprocol 1

### First Run

To an 8 L hydrogenator (equipped with an overhead magnetic stir drive, internal solenoid cooling coil, gas inlet valve and sampling valve) was added 10% Pd/C catalyst (50 wt % water; Degussa type E101 NE/W catalyst; 33 g, 15 6 mmol palladium) followed by a solution of isopropyl acetate (2.4 L) and isopropyl alcohol (1 L), and 2-ethylhexanoic acid (21 g, 146 mmol). The mixture was sparged with a stream of N₂ through the mixture for 5 min. The mixture was agitated and the vessel was pressurized to 400 psi with N₂, then vented to 50 psi. The vessel was pressurized to 400 psi with N₂, again, and the mixture was agitated for 20 min. The vessel was vented to 100 psi and was then pressurized to 1000 psi with hydrogen. The mixture was vigorously stirred (80% power) under an H₂ atmosphere for 30 min., then vented to atmospheric pressure. Furan 39 (230 g, 625 mmol) was added in one portion, as a solid, and the vessel was pressurized to 350 psi with N₂. The vessel was vented to 50 psi, then pressurized to 1130 psi with hydrogen. The mixture was heated to 105° C. and the mixture was stirred and the pressure was maintained at 120-1310 psi H₂. The mixture was sampled to monitor the course of the reaction. After 26 hours, the vessel was cooled to 25° C., vented, and additional 10% Pd/C (25 g, pre-hydrogenated in 150 mL isopropyl acetate) was added. The mixture was pressurized with H₂ and heated. The mixture was vigorously agitated at 107° C. under 1230 psi H₂. After 44 hours the reaction was complete based on LCMS analysis. The vessel was cooled to 21° C. and was vented. The vessel was pressurized to 400 psi with N₂ and the mixture was vigorously stirred for 40 min. then vented. The reaction mixture was filtered through a 2 L frit funnel (medium frit) containing a bed of Celite® 545 filter material (218 g, pre-washed with isopropyl acetate) topped with Whatman No. 1 filter paper. The Celite® filter material was washed twice with 2:1 isopropyl acetate-isopropanol (2×250 mL), under vacuum, being careful not to allow the top Pd/C layer to become dry. The top Pd/C layer was removed with a spatula and the residual solvents were removed from the cake by applying full vacuum. The combined filtrate and washes were concentrated in vacuo, removing 3.3 L of solvent. The resulting viscous solution (about 600 mL) was polish filtered through Whatman No. 1 filter paper and diluted with heptane (1 L). The solution was concentrated in vacuo to give a thick slurry, which was diluted with additional heptane (1.5 L). The slurry was heated to 50° C. and was gradually cooled to 15° C. over 1 hour. The slurry was vacuum filtered through a Buchner funnel and was washed twice with heptane (2×200 mL). The filter cake was dried in a vacuum oven (16 h, 50° C.) to give 53.3 g of terameprocol 1 (24% purity>99% as determined by GC) ¹H NMR (CDCl₃, 400 MHz) δ 0.85 (d, 6H, J=6.6 Hz), 1.83-1.92 (m, 2H), 2.30 (dd, 2H, J=9.3, 13.5 Hz), 2.76 (dd, 2H, J=5.0, 13.5 Hz), 3.85 (s, 6H), 3.86 (s, 6H), 6.65 (d, 2H, J=2.0 Hz), 6.70 (dd, 2H, J=8.0, 2.0 Hz), 6.79 (d, 2H, J=8.0 Hz). ¹³C NMR (CDCl₃, 200 MHz) δ 16.19, 38.80, 39.14, 55.76, 55.86, 110.99, 112.22, 120.90, 134.42, 147.02, 148.68. LCMS (m/z=358.9).

### Example 5

### Preparation of Terameprocol 1

### Second Run

To an 8 L hydrogenator, equipped with an overhead magnetic stir drive and heating mantle, were charged a finely divided mixture of 78.65 g (32.6 mmol) 10R39 10% Pd/C (55.9% wet) catalyst (available through Johnson-Matthey Inc.) and 11.55 g (5.4 mmol) Degussa E101 10% Pd/C (50% wet) catalyst (available from Sigma-Aldrich Inc.). To the vessel was charged n-propyl acetate (3.74 L) and the vessel was pressurized to 800 psi with H₂. The mixture was stirred at the maximum stir speed at room temperature for 30 min. to 1 hour. The vessel was vented to atmospheric pressure, the lid opened under N₂ atmosphere and a slurry of furan 39 (400 g, 108 mmol) in n-propyl acetate (1.86 L was charged to the vessel). The mixture was heated to 100° C. under 1000 psi H₂ pressure at maximum stir speed. The reaction was monitored by HPLC until all furan 39 is consumed and less than 2% THF intermediate 40 is present. The reaction mixture was cooled to 20-25° C. and the vessel was vented to atmospheric pressure. The reactor lid was removed and the reaction mixture was sparged with N₂. Immediately, the reaction mixture was filtered through a bed of Celite®545 filter material (800 g) and the Celite® filter material cake was washed with n-propyl acetate (4 L). The combined n-propyl acetate filtrate was washed with water (2 L), 5 wt % aqueous potassium carbonate solution (2 L) and brine (2 L). The organic stream was dried over Na₂SO₄ (400 g), filtered, then solvent was removed in vacuo at 50° C. The resulting residue was diluted with heptane (2 L) and solvent was removed in vacuo at 50° C. The resulting solids were suspended in 15% (v/v) IPA-heptane (1.6 L), heated to 50-60° C. and cooled to 20° C. over 1 hour. The slurry was agitated for 1 hour at 20° C. and vacuum filtered (up to 18 hours). The crude solids were transferred (289 g) to a 2 L vessel, equipped with an overhead stir drive, condenser and heating mantle, and 15% (v/v) IPA-heptane (578 mL) was added. The mixture was heated to 65° C. until the slurry thinned (about 5 min.) and the mixture was allowed to cool to 15° C. over 3.5 hours. The slurry was vacuumed and the cake was washed with chilled (5-10° C.) 15% (v/v) IPA-heptane (300 mL). The solids were dried in vacuo to constant weight (217 g, 55.9% yield, purity>99% as determined by GC). Analytical data were identical with that shown in Example 4.

### Example 6

### Preparation of Terameprocol

### Third Run

400 g of furan 39 were hydrogenated in n-propyl acetate using 10R39 catalyst (2.5 mol %) and E101 NE/W GG (0.5 mol %) at 100° C. After pre-reducing the catalyst mixture at room temperature, the reactor lid was opened and substrate was added as a solid. The mixture was heated under H₂ pressure and monitored by HPLC. The reaction scheme is shown in Scheme 25 and the reaction profile is shown in Table W, below. Within 3 hours, the furan was completely converted to THF intermediate 40 and products (Table W, entry 3). The amount of THF intermediate 40 steadily decreased over the next 2 hours (Table W, entries 4-5), and the mixture was cooled to room temperature and held for 12 h (Table 22, entry 6). Water (10% by volume of n-PrOAc) was then added and the mixture was heated under H₂ pressure. After an additional 4 hours of heating under H₂ pressure, HPLC analysis showed low levels of THF intermediate 40 (Table W, entry 9).

TABLE W

| | Time | T | P | Furan | THF Int | Teramepl | Impurity |
|---|---|---|---|---|---|---|---|
| | (h) | (° C.) | (psi) | 39 (%) | 40 (%) | 1 (%) | 48 (%) |
| 1 | 1 | 100 | 960 | NA | NA | NA | NA |
| 2 | 2 | 100 | 980 | 18.2 | 29.5 | 38.4 | 13.9 |
| 3 | 3 | 100 | 980 | 0.0 | 24.7 | 55.4 | 19.9 |
| 4 | 4 | 100 | 980 | 0.0 | 17.1 | 61.2 | 21.7 |
| 5 | 5 | 100 | 980 | 0.0 | 11.8 | 64.7 | 23.5 |
| 6 | 6-18 | 23 | 980-600 | Held without stirring, water | | | |
| | | | | added at t = 18 hours | | | |
| 7 | 18 | 23 | 600 | 0.0 | 9.5 | 64.1 | 26.4 |
| 8 | 21 | 100 | 950 | 0.0 | 4.3 | 67.9 | 27.8 |
| 9 | 22 | 100 | 840 | 0.0 | 2.8 | 67.1 | 30.1 |

| | | | | | |
|---|---|---|---|---|---|
| 10 | Isolated Product | 0.0 | 1.2 | 98.2 | 0.6 |
| | (46.6% yield) | | | | |

After filtering the mixture through Celite® filter material and washing the filter cake with additional n-propyl acetate, the product stream underwent aqueous work-up. The solvent was evaporated and the residual n-propyl acetate was chased with heptane. Terameprocol was then crystallized from heptane. The resulting sticky solids were difficult to manipulate and a spatula was used to free the solids from the walls of the vessel. After filtration, three heptane washes were used to remove the residual cyclized impurity 48 from terameprocol. After drying in vacuo at 50° C. Terameprocol was isolated in 46.6% yield, with 98.2% purity (by HPLC) (Table W, entry 10). Analytical data were identical with that shown in Example 4.

### Example 7

### Preparation of Terameprocol 1

### Fourth Run

In this experiment, several modifications were made in comparison with Example 6. First, for safety reasons, furan 39 was added to the pre-hydrogenated catalyst as a slurry instead of a powder to prevent excessive hydrogen off-gassing during substrate addition. Second, since the conversion of THF intermediate 40 to product did not appear to stall, the hydrogenation was allowed to proceed without the addition of water. Third, the work-up involved crystallization from isopropanol (IPA)-heptane to improve product handling.

The reaction profile for this fourth run, using 400 g furan 39, 2.5 mol % 10R39 catalyst, and 0.5 mol % Degussa E101 catalyst, is shown in Table X. The hydrogenation was started in the evening and allowed to stir under H₂ pressure at 100° C. overnight. During this time, the internal pressure dropped from 960 psi to 300 psi due to a leak in the vessel and 15.8% starting furan was present (Table X, entry 1). The vessel was pressurized and, after 3 hours, all the furan was consumed and only 2.5% THF intermediate 40 remained (Table X, entry 2). Without the addition of water, THF intermediate 40 content reached 1.1% between 17-23 hours (Table X, entry 3). Heating was stopped and the hydrogenator was vented after 25 hours. The reaction mixture was held for an additional 18 hours prior to filtration. Since water was not added during the reaction, it was introduced prior to filtration to prevent the catalyst from sticking to the internal cooling coils and walls of the hydrogenation vessel.

TABLE X

| | Time | T | P | Furan | THF Int | Teramepl | Impurity |
|---|---|---|---|---|---|---|---|
| | (h) | (° C.) | (psi) | 39 (%) | 40 (%) | 1 (%) | 48 (%) |
| 1 | 14 | 100 | 300 | 15.8 | 4.4 | 50.1 | 29.7 |
| 2 | 17 | 100 | 800 | 0.0 | 2.5 | 63.3 | 34.2 |
| 3 | 23 | 100 | 980 | 0.0 | 1.1 | 67.1 | 31.8 |

| | | | | | |
|---|---|---|---|---|---|
| 4 | Isolated Product¹ | 0.0 | 0.0 | 99.1 | 0.2 |
| | (39.4% yield) | | | | |

¹0.1% new impurity (Retention time: 1.03 min) formed during crystallization from IPA-heptane

A modification was also made in the product isolation. After aqueous work-up and solvent evaporation, the waxy solid product mixture was dissolved in 3:1 heptane-IPA (3 mL/g input) at 80° C. Terameprocol crystallized while cooling to room temperature, generating a uniform slurry. Unlike the product slurry obtained from run 3 (Example 6), which was waxy and difficult to manipulate, the uniform slurry of this Example 7 generated in run 4 was easily transferred and filtered. The cake was washed twice with heptane and dried in vacuo at 50° C. Terameprocol 1 was isolated in 39.4% yield and 99.1% purity. Notably, the IPA-heptane effectively removed the residual THF intermediate 40. Unexpectedly, however, a new unknown impurity was formed during the crystallization step. It is believed that terameprocol might not be stable in the presence of IPA at higher temperatures. As a corrective measure the product isolation in the next run 5 (Example 8) used less IPA and involved a re-slurry at lower temperatures instead of a recrystallization at higher temperatures. Analytical data were identical with that shown in Example 4.

### Example 8

### Preparation of Terameprocol 1

### Run 5

A major modification was made to improve process safety in this fifth run, compared to runs 3 and 4 of Examples 6 and 7, respectively. In this experiment, the catalysts, substrate and solvent were added to the vessel and the mixture was pre-hydrogenated at room temperature prior to heating. Prior to scale-up, a 10 g scale front run in a 1 L hydrogenation vessel showed only 3.6% THF intermediate 40 after 18 hours and was not expected to be a problem on a larger scale.

Unfortunately, the reaction behaved differently on the scale in the Example as shown in Table Y, using 400 g furan 39, 2.5 mol % 10R39 and 0.5 mol % Degussa E101 catalyst, monitored by HPLC. The conversion of furan 39 to THF intermediate 40 was sluggish (Table Y, entries 2-3), suggesting the need for pre-hydrogenation in the absence of substrate. After 25 hours, despite a significant drop in pressure, all the furan 39 was consumed and 4% of the THF intermediate 40 remained. At this point, the mixture was held over the weekend at room temperature and atmospheric pressure (Table Y, entry 4). During the hold period, the amount of THF intermediate 40 decreased to 2.3% (Table Y, entry 5). The mixture was then subjected to work-up conditions as described in example 4.

TABLE Y

| | Time | T | P | Furan | THF Int | Teramepl | Impurity |
|---|---|---|---|---|---|---|---|
| | (h) | (° C.) | (psi) | 39 (%) | 40 (%) | 1 (%) | 48 (%) |
| 1 | 14 | 100 | 200 | NA | NA | NA | NA |
| 2 | 16 | 100 | 920 | 51.6 | 3.4 | 31.4 | 13.6 |
| 3 | 25 | 100 | 100 | 0.0 | 4.0 | 64.8 | 31.2 |
| 4 | 26-89 | 20 | 0 | Held without stirring | | | |
| 5 | 89 | 20 | 0 | 0.0 | 2.3 | 66.0 | 31.7 |

| | | | | | |
|---|---|---|---|---|---|
| 6 | Isolated Product | 0.0 | 0.0 | 99.5 | 0.1 |
| | (36.6% yield)¹ | | | | |

¹<0.1% new impurity (Retention time: 1.03) formed during crystallization from IPA-heptane

After filtering the product mixture through Celite® filter material, the organic stream was significantly discolored. The typically clear to faint yellow solution was dark yellow to orange. Washing the organic stream with water did little to remove color. However, the aqueous potassium carbonate wash became orange and removed highly colored, presumably acidic, impurities. After evaporating solvent, the resulting waxy solids were slurried in 20% IPA-heptane (2.75 mL/g) at 60° C. The slurry was cooled to room temperature and held for 3 hours. The resulting uniform slurry was filtered and washed twice with heptane. After drying in vacuo at 50° C., Terameprocol was isolated in 36.6% yield. The change in product isolation had a positive impact on purity (Table Y, entry 6). Again, the THF intermediate 40 was effectively removed. In addition, the new impurity that formed during the IPA-heptane reslurry was reduced when less IPA was used and when the temperature was decreased from 80° C. to 60° C. The lower yield was attributed to the extended hold time on acidic carbon.

### Example 9

### Preparation of Terameprocol 1

### Rework Procedure

To improve product quality, a re-work was developed based on the IPA-heptane recrystallization. A 150 g sample of terameprocol (75 g from run 3 of Example 6, 75 g from run 4 of Example 7) was slurried in 15% IPA-heptane (4 mL/g input). The slurry was warmed to 60° C. and held for 20 min. The resulting thin slurry was gradually cooled to 10° C. over 100 min. and was immediately filtered. After de-liquoring, the cake was washed once with cold heptane (10° C.; 1 mL/g input). Terameprocol 1 was isolated with 94.8% recovery, after drying in vacuo at 50° C., with 99.52% purity (compared to an average 98.15% purity of the input terameprocol). Unfortunately, the rework did little to remove the THF impurity 40, as it was present in 0.48% (compared to 0.60% in the input terameprocol). Analytical data were identical with that shown in Example 4.

### Example 10

### Preparation of Terameprocol 1

### Sixth Run

In this reaction, several modifications based on the results in Examples 6-9 were implemented in order to optimize the yield and purity of terameprocol. First, to avoid a slower conversion, consistent H₂ pressure was maintained at 1000 psi during the reaction. Second, to improve conversion of starting material to products, the loading of 10R39 catalyst was increased from 2.5 mol % to 3 mol %. Third, in order to avoid unnecessary losses due to either product absorption onto carbon or decomposition under acidic conditions, the reaction mixture was filtered through Celite® filter material immediately upon reaction completion. Finally, to avoid losses during product isolation the product was initially isolated from 15% IPA-heptane, then reslurried with IPA-heptane instead of washing several times with heptane.

After pre-reducing the catalyst, the vessel was opened and a slurry of furan 39 (400 g) in n-propyl acetate was added. The mixture was heated and the pressure was carefully monitored to ensure consistent pressure (1000 psi) during the course of the reaction. The results for this run, using 3.0 mol % 10R39 catalyst and 0.5 mol % Degussa E101 catalyst, monitored by HPLC, are shown in Table Z. After 3 hours, complete consumption of furan 39 was observed by HPLC (entry 1). Within the next 5 hours, the THF intermediate 40 was steadily converted to products (entries 2-4). After 8 hours, the reaction mixture was cooled to room temperature and immediately filtered through Celite® filter material. The organic stream was held overnight at room temperature.

TABLE Z

| | Time | T | P | Furan | THF Int | Teramepl | Impurity |
|---|---|---|---|---|---|---|---|
| | (h) | (° C.) | (psi) | 39 (%) | 40 (%) | 1 (%) | 48 (%) |
| 1 | 3 | 100 | 1000 | 0.0 | 21.4 | 58.5 | 20.1 |
| 2 | 5 | 100 | 1000 | 0.0 | 5.6 | 69.1 | 25.3 |
| 3 | 7 | 100 | 1000 | 0.0 | 2.8 | 70.2 | 26.9 |
| 4 | 8 | 100 | 1000 | 0.0 | 1.2 | 70.6 | 28.2 |

| | | | | | |
|---|---|---|---|---|---|
| 5 | Isolated Product | 0.0 | 0.0 | 99.0 | 0.5 |
| | (55.9% yield)¹ | | | | |

¹0.2% new impurity (Retention time: 1.03) formed during crystallization from IPA-heptane

After the overnight hold, the organic stream underwent a purification procedure described in example 4 to give waxy solids, which were then slurried in 15% IPA-heptane (4 mL/g input) at 55° C. for 15 min. and the resulting uniform slurry was cooled to 20° C. over 1 hour. After an additional 1 hour of stirring at 20° C., the slurry was filtered. Instead of immediately washing the cake with heptane, the cake was allowed to dry on the vacuum funnel The resulting crude cake (289.58 g), was transferred to a 2-L vessel and suspended in 15% IPA-heptane (2 mL/g). The suspension was heated to 65° C. and held until the slurry thinned (5 min.). The slurry was cooled to 15° C. over 4 hours, vacuum filtered, then washed once with chilled 15% IPA-heptane (1 mL/g, cooled to 5-10° C.). After drying in vacuo at 70° C., terameprocol was isolated in 55.9% yield, with 99.0% purity (by HPLC). Analytical data were identical with that shown in Example 4.

What is claimed is:

1. A manufacturing process for making terameprocol (1) which comprises the following reaction scheme, wherein a first general reaction is the formation of a furan intermediate (39) and a second general reaction is the ring-reduction and ring-opening of the furan intermediate (39) to form the terameprocol (1):

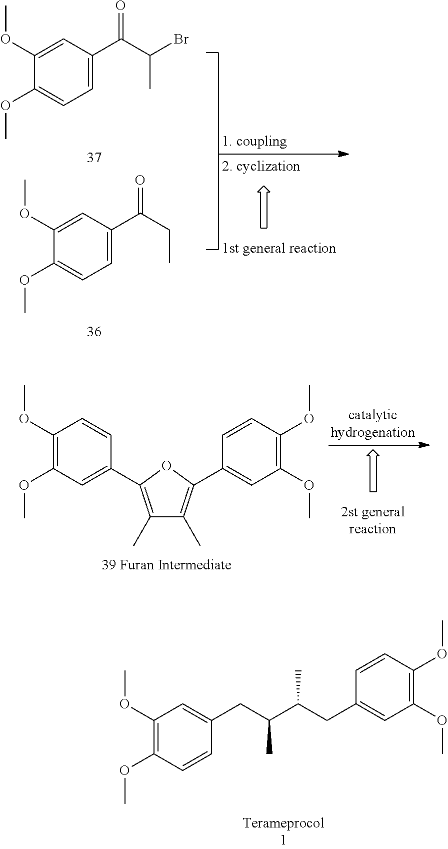

2. The process of claim 1, wherein the first general reaction to form the furan intermediate (39) is a two-reaction, one-purification process, in which the first reaction is a coupling reaction, in which a ketone-catechol compound (36) is treated by an organic basic catalyst, followed by reaction with a bromide-ketone-catechol compound (37) to give a corresponding diketone intermediate, and in which the second reaction is a cyclization reaction, in which the diketone intermediate is converted to the furan intermediate (39).

3. The process of claim 2, wherein the organic basic catalyst for the coupling reaction of the ketone-catechol compound (36) with the bromide-ketone-catechol compound (37) is an alkali metal salt of an alkyl alcohol having a formula MOR, in which M is an alkali metal ion selected from the group consisting of K⁺, Na⁺ and Li⁺, and R is a linear or branched saturated hydrocarbon chain having 4 to 10 carbon atoms; the amount of the basic catalyst used is about 0.5 to about 1.5 molar equivalents of compound (36); the molar ratio of compound (37) to compound (36) is about 0.5 to about 1.7; and a solvent system is used tin the coupling reaction, wherein the solvent system is a single solvent or a mixture of two solvents selected from the group consisting of tetrahydrofuran, 1,2-dimethoxyethane, 1,3-dimethoxypropane, and dimethyl formamide.

4. The process of claim 2, wherein the reaction temperature for the coupling reaction is about −30° C. to about −70° C., and the temperature for the cyclization reaction is about 55° C. to about 65° C.

5. The process of claim 1, wherein the catalyst for the second general reaction is a mixture of two types of palladium catalysts, one being favorable for furan ring-reduction, and the other being favorable for a ring-opening reaction.

6. The process of claim 5, wherein the palladium catalysts contain about 40 to about 60% water, and on a dry basis, about 5% to about 20% palladium, and about 80% to about 95% active carbon, or silica gel, or alumina.

7. The process of claim 6 wherein the palladium catalyst is selected from at least one of the following: 10% Pd on carbon (cat.# A5011023, from Johnson Matthey Company); 5% Pd on SiO₂-Al₂O₃ (cat# C-7079, from Johnson Matthey Company); 10% Pd on carbon (cat.# E101023, from Johnson Matthey Company), 10% Pd on carbon (cat.# 10R374, from Johnson Matthey Company), 10% Pd on carbon (cat.# 10R490, from Johnson Matthey Company), 10% Pd on carbon (cat.# 10R37, from Johnson Matthey Company), 10% Pd on carbon (cat.# E101GG, from Sigma-Aldrich), 10% Pd on carbon (cat.# A402032, from Johnson Matthey Company). Examples of catalysts, which are favorable for the ring-opening are 10% Pd on carbon (cat.# A402028-10, from Johnson Matthey Company). 10% Pd on carbon (cat.# 10R39, from Johnson Matthey Company), 10% Pd on carbon (cat.# 20R91, from Johnson Matthey Company), 10% Pd on carbon (cat.# E101 MLP, from Aldrich), 10% Pd on carbon (cat.# A470201-10, from Johnson Matthey Company), 10% Pd on carbon (cat.# 10R90, from Johnson Matthey Company).

8. The process of claim 1, wherein the second general reaction involves a catalyst present in an amount of about 2 mol % to about 4 mol % Pd based on the amount of the furan intermediate (39); the pressure for the second general reaction is about 60 bar to about 100 bar; the solvent is n-butyl acetate, isopropyl acetate or isopropanol; and the reaction temperature of the second general reaction is about 80° C. to about 110° C.

9. A manufacturing process for a furan intermediate (39) which comprises the following reaction scheme:

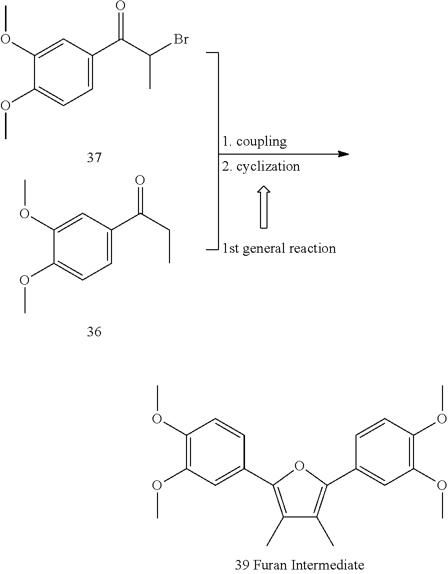

10. The process of claim 9, wherein the first general reaction to form the furan intermediate (39) is a two-reaction, one-purification process, in which the first reaction is a coupling reaction, in which a ketone-catechol compound (36) is treated by an organic basic catalyst, followed by reaction with a bromide-ketone-catechol compound (37) to give a corresponding diketone intermediate, and in which the second reaction is a cyclization reaction, in which the diketone intermediate is converted to the furan intermediate (39).

11. The process of claim 10, wherein the organic basic catalyst for the coupling reaction of the ketone-catechol compound (36) with the bromide-ketone-catechol compound (37) is an alkali metal salt of an alkyl alcohol having a formula MOR, in which M is an alkali metal ion selected from the group consisting of K⁺, Na⁺ and Li⁺, and R is a linear or branched saturated hydrocarbon chain having 4 to 10 carbon atoms; the amount of the basic catalyst used is about 0.5 to about 1.5 molar equivalents of compound (36); the molar ratio of compound (37) to compound (36) is about 0.5 to about 1.7; and a solvent system is used in the coupling reaction, wherein the solvent system is a single solvent or a mixture of two solvents selected from the group consisting of tetrahydrofuran, 1,2-dimethoxyethane, 1,3-dimethoxypropane, and dimethyl formamide.

12. The process of claim 10, wherein the reaction temperature for the coupling reaction is about −30° C. to about −70° C., and the temperature for the cyclization reaction is about 55° C. to about 65° C.

13. A manufacturing process for making terameprocol (1) which comprises the ring-reduction and ring-opening of a furan intermediate (39) to form the terameprocol (1):

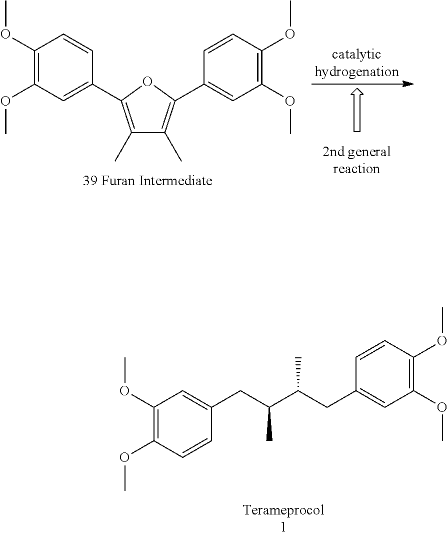

14. The process of claim 13, wherein the catalyst for the second general reaction is a mixture of two types of palladium catalysts, one being favorable for furan ring-reduction and the other being favorable for a ring-opening reaction.

15. The process of claim 14, wherein the palladium catalysts contain about 40 to about 60% water, and on a dry basis, about 5% to about 20% palladium, and about 80% to about 95% active carbon, or silica gel, or alumina.

16. The process of claim 14, wherein the reaction involves a catalyst present in an amount of about 2 mol % to about 4 mol % Pd based on the amount of the furan intermediate (39); the pressure for the second general reaction is about 60 bar to about 100 bar; the solvent is n-butyl acetate, isopropyl acetate or isopropanol; and the reaction temperature of the second general reaction is about 80° C. to about 110° C.
